(19) United States
(12) Patent Application Publication
(10) Pub. No.: US 2014/0186861 A1
(43) Pub. Date: Jul. 3, 2014

(54) SOLUBLE TREPONEMA PALLIDUM PROTEIN TP0453, TP0453-TP0326 FUSION PROTEIN, AND USE IN SYPHILIS DIAGNOSIS

(71) Applicants: Caroline E. Cameron, Victoria (CA); Martin J. Boulanger, Victoria (CA); Brenden C. Smith, Abbotsford (CA)

(72) Inventors: Caroline E. Cameron, Victoria (CA); Martin J. Boulanger, Victoria (CA); Brenden C. Smith, Abbotsford (CA)

(73) Assignee: UVic Industry Partnerships Inc., Victoria, BC (CA)

(21) Appl. No.: 14/127,568

(22) PCT Filed: Jul. 10, 2012
(86) PCT No.: PCT/US2012/046040

§ 371 Date: Dec. 19, 2013

Related U.S. Application Data
(60) Provisional application No. 61/506,352, filed on Jul. 11, 2011.
(60) Provisional application No. 61/602,151, filed on Feb. 23, 2012.

(51) Int. Cl. C07K 14/20; G01N 33/569

(52) U.S. Cl. CPC C07K 14/20; G01N 33/56911
USPC 435/7.92; 530/350; 536/23.7; 530/387.9; 435/69.3; 435/320.1; 435/252.33; 435/254.2; 435/348; 422/69; 435/287.2; 436/501

(57) ABSTRACT

The present application provides methods of producing soluble Treponema pallidum protein Tp0453 (such as the fragment shown in SEQ ID NO: 3). For example, the protein can be expressed from the pET28a vector in a cell and the resulting soluble Tp0453 protein isolated from the cell. Also provided are the isolated soluble Tp0453 protein, as well as Tp0453-Tp0326 chimeric proteins (e.g., SEQ ID NO: 11), and methods of using the proteins to detect antibodies specific for T. pallidum subsp. pallidum, for example to diagnose syphilis. Devices and kits that incorporate Tp0453 protein and Tp0453-Tp0326 chimeric proteins, such as lateral flow devices, are also disclosed. Kits that include the soluble Tp0453 protein and kits that include solid substrates containing the soluble Tp0453 protein are also provided.

### CROSS-REFERENCE TO RELATED APPLICATIONS

This application claims priority to US Provisional Application No. 61,506,352 filed Jul. 11, 2011 and U.S. Provisional Application No. 61/602,151 filed on Feb. 23, 2012, both herein incorporated by reference in their entirety.

### FIELD

The application relates to a fragment of the Tp0453 protein and methods for generating a soluble form of the Tp0453 protein fragment, Tp0453-Tp0326 chimeric proteins, and their use to detect Treponema pallidum subsp. pallidum antibodies, for example to diagnose syphilis. The technology relates in some examples to a 256 amino acid portion of a recombinant Tp0453 protein that has been solubilised by expression from the pET28a vector.

### BACKGROUND

Syphilis is a chronic infection caused by the spirochete Treponema pallidum subsp. pallidum that is generally transmitted either through sexual contact or vertically from an infected mother to her fetus. Syphilis is a global health concern with an estimated burden of 25 million people worldwide and an estimated annual new incidence rate of 12 million cases. Further, infection with T. pallidum has been shown to increase the likelihood of contracting HIV. Congenital syphilis is recognized as the most significant disease affecting pregnancies and newborns worldwide, with over 2 million pregnant women estimated to be infected with syphilis every year. Without treatment, there are adverse outcomes in 69% of cases, including spontaneous abortion or stillbirth, neonatal complications, and infant mortality.

As a multi-stage disease, syphilis has historically been called the great imitator due to the similarity of its symptoms to other diseases. Primary syphilis is characterized by a painless open sore called a chancre, which develops on average 3 weeks after infection at the site of inoculation. The chancre spontaneously resolves, and 1-3 months later secondary symptoms may present. Secondary infection typically manifests as a generalized rash, often localizing to the trunk of the body, palms of the hands, and soles of the feet. After 1-3 months, secondary symptoms resolve and the disease enters an asymptomatic latent phase. In some instances the disease can progress from latency to a tertiary stage, which can involve the development of gummas, central nervous system complications or cardiovascular disease. Since the symptoms of syphilis infection are so similar to other diseases, and resolve on their own, syphilis has always been a challenging disease to diagnose clinically.

During primary infection when a chancre is present, dark-field microscopy and/or PCR can be performed to identify spirochetes present at the site of infection. The chancre normally resolves in 4-6 weeks, and often goes unnoticed if internally located in either the anus or vagina, making diagnosis by dark-field microscopy extremely limited. This method of diagnosis also requires the presence of both a dark-field microscope and a trained microscopist, which further limits the usefulness of this laboratory-based diagnostic method. The gold standard for syphilis diagnosis relies on the use of a series of serological testing regimes. Traditional serological testing algorithms for diagnosis of T. pallidum infection comprise detection of nontreponemal antibodies using the rapid plasma reagin (RPR) test or Venereal Disease Research Laboratory (VDRL) test, followed by further screening of reactive samples for detection of treponemal-specific antibodies using the fluorescent treponemal antibody-absorption (FTA-ABS) test, the microhemagglutination assay for T. pallidum (MHA-TP) test or the T. pallidum particle agglutination (TP-PA) test. The necessity for multiple tests is due to the inadequacies of current syphilis diagnostic tests. The RPR and VDRL show median sensitivities of only 86% and 78%, respectively, during primary-stage infections and 73% and 71%, respectively, for late-stage infections. Further, these nontreponemal tests have been shown to exhibit cross-reactivity against a multitude of diseases and health conditions, including chickenpox, rheumatoid arthritis, pregnancy and advanced age. The confirmatory tests (MHA-TP, TP-PA and FTA-ABS) are expensive and have major limitations due their reliance on experienced technicians and adequate testing facilities, factors that are especially restricting in rural areas and developing countries. These confirmatory tests also have poor sensitivities for detecting early infection, with 88% reported for the MHA-TP, 88% for the TP-PA, and 84% for the FTA-ABS.

The relatively recent introduction of automated enzyme immunoassays (EIAs) and chemiluminescence immunoassays (CIAs) for diagnosing syphilis infection has prompted implementation of a reverse algorithm screening protocol, whereby samples are first screened via EIA/CIA against a panel of recombinant treponemal proteins [one or more of TpN15 (Tp0171), TpN17 (Tp0435), TpN47 (Tp0574), and TpN44 (Tp0768; TmpA)], followed by re-screening of positive samples with a nontreponemal test and discordant samples (e.g., EIA/CIA reactive and RPR/VDRL nonreactive) with a treponemal test. This testing algorithm sequence has been observed in three studies to lead to discordant result rates of over 50%. Further, this reverse testing algorithm has been observed to lead to a higher false-reactive rate than the traditional screening algorithm in populations with a low prevalence of syphilis. Rapid point-of-care (POC) tests based upon the same panel of recombinant proteins have also become available, although a recent review of results from 15 studies, undertaken at both sexually transmitted infection and antenatal clinics, showed an average sensitivity of only 86%. The poor performance of syphilis EIA, CIA and POC tests is thought to be primarily due to inadequacies associated with the recombinant proteins currently being used in these diagnostic assays.

Proteins that are essential for proper bacterial function have been a challenge to identify because T. pallidum cannot be cultured in vitro. Proteins located on or near the surface of bacteria are of extreme interest because they either directly facilitate pathogenesis or provide the structural or functional framework for surface proteins. Soluble protein is necessary for diagnostic antigen incorporation into all diagnostic test formats.

Preliminary studies have identified three T. pallidum proteins which exhibit greater sensitivity and specificity than current diagnostic tests, especially during early-stage infections when the risk of syphilis transmission is at its highest.

Previous attempts by others have been unsuccessful to achieve soluble expression of both full length Tp0453 and Tp0453 fragments. Efforts thus far have included cloning into multiple expression vectors. To date it has been possible to partially solubilise full length and Tp0453 fragments using detergents or other denaturing agents such as urea, but these additions can affect downstream applications of the protein and therefore it is valuable to achieve solubility by another means. Tp0453 is the most sensitive and specific syphilis antigen known at this time. For example, Van Voorhis et al. (J. Clin. Microbiol. 41:3668-74, 2003; herein incorporated by reference) teaches the sensitivity and specificity of six recombinant T. pallidum proteins, including Tp0453. As shown in Van Voorhis et al., Tp0453 has 100% sensitivity and 100% specificity in syphilis serodiagnosis. However, improved methods for solubilizing Tp0453 are needed.

### SUMMARY

Due to limitations inherent in the current diagnostic tests, a new direction is needed for the diagnosis of syphilis infection. Dark-field microscopy (DFM) is impractical due to the difficulty in identifying internal chancres, as well as the necessity of a dark-field microscope, and trained microscopist (Ratnam, 2005. Can J Infect Dis Med Microbiol 16:45-51). The gold standard for most testing facilities is the non-treponemal, anti-cardiolipin tests (RPR and VDRL), yet they exhibit poor sensitivities and specificities. These non-treponemal tests are especially poor at diagnosing primary (78-86%) and late latent (71-73%) infection, and have been shown to exhibit cross-reactivity against numerous diseases and health conditions (including chickenpox, rheumatoid arthritis, pregnancy, and advanced age) (Larsen et al., 1995. Clin Microbiol Rev 8:1-21; Ratnam, 2005. Can J Infect Dis Med Microbiol 16:45-51). The confirmatory tests (TPPA and MHA-TP) also have poor sensitivities for early infection (88% for the TPPA) and exhibit cross-reactivity with serum from patients who have unrelated medical conditions (including relapsing fever, cirrhosis, pregnancy and advanced age) (Ratnam, 2005. Can J Infect Dis Med Microbiol 16:45-51). These treponemal tests are expensive, since they rely on crude extracts of T. pallidum grown in a rabbit model, and processing requires experienced technicians. EIA and CIA tests based on recombinant treponemal proteins (one or more of TpN15, TpN17, TpN47, and TmpA) have been developed; however, studies have shown discordant result rates of over 50% (Peterman et al., 2008. MMWR 57:872-875); Radolf et al., 2011. MMWR 60:133-137). Rapid point-of-care (POC) tests based on the same panel of recombinant proteins showed average sensitivities of only 86% (Tucker et al., 2010. Lancet Infect. Dis. 10:381-386). The poor performance of syphilis EIA, CIA and POC tests is thought to be primarily due to inadequacies associated with the recombinant proteins currently being used in these diagnostic assays.

Thus provided herein are isolated proteins that can be used in a sensitive and specific rapid point-of-care (POC) test, which can in some examples be an EIA or CIA. A soluble form of Tp0453 (amino acids A32-S287 of the native protein) and a Tp0453-Tp0326 chimeric protein showed high sensitivity, especially in primary cases—a major limitation of current diagnostic tests, and were extremely specific, even when tested against potentially cross-reactive sera.

A Tp0453 protein fragment (e.g., SEQ ID NO: 3) and Tp0453-Tp0326 chimeric protein (e.g., SEQ ID NOS: 10 and 11), and variants thereof, (and nucleic acid molecules encoding such proteins) are provided. For example, the disclosure provides isolated Tp0453-Tp0326 chimeric proteins that include the structure X-Y or Y-X, wherein X comprises a soluble fragment of a Tp0453 protein (e.g., an amino acid sequence comprising the amino acid sequence shown in SEQ ID NO: 3, or a sequence comprising at least 90% sequence identity to SEQ ID NO: 3, for example a protein having 0 to 20 conservative amino acid substitutions) and wherein Y comprises a soluble fragment of a Tp0326 protein (e.g., an amino acid sequence comprising the amino acid sequence shown in SEQ ID NO: 9, or a sequence comprising at least 90% sequence identity to SEQ ID NO: 9, for example a protein having 0 to 20 conservative amino acid substitutions). The Tp0453-Tp0326 chimeric protein can further include a purification tag (for example at the N-terminus or C-terminus), and/or a linker between the Tp0453 and Tp0326 fragments.

In addition, methods of producing soluble Tp0453 protein and Tp0453-Tp0326 chimeric proteins are provided. For example, the method can include expressing a Tp0453 protein or Tp0453-Tp0326 chimeric protein from a pET28a vector in a cell and then isolating the expressed soluble protein from the cell. In one example, the expressed soluble protein is isolated from the cell by lysing the cell and isolating the protein from the resulting cell lysate. The disclosure also provides isolated soluble Tpo453 protein and Tp0453-Tp0326 chimeric protein produced by the disclosed methods.

The disclosure also provides isolated soluble Tp0453 proteins and Tp0453-Tp0326 chimeric proteins produced by the disclosed methods, as well as kits containing such soluble proteins. The isolated soluble Tp0453 proteins generated using the disclosed methods can be used to produce antibodies, such as monoclonal or polyclonal antibodies. The isolated soluble Tp0453 proteins and Tp0453-Tp0326 chimeric proteins generated using the disclosed methods can also be used to diagnose syphilis.

The disclosed isolated soluble Tp0453 proteins and Tp0453-Tp0326 chimeric proteins can also be part of an immunoassay device, such as an ELISA plate, a lateral flow device, or microfluidic device, wherein the device permits detection of T. pallidum antibodies. Such devices are also provided herein, as are kits containing such devices. In some examples, such immunoassay devices can include other diagnostic antigens or antibodies, such as antigens associated with a sexually transmitted infection (such as HIV antigens or antibodies), TB antigens or antibodies, lipoidal antigens or antibodies, or combinations thereof.

Methods of detecting Treponema pallidum subsp. pallidum antibodies using the disclosed soluble Tp0453 protein and/or Tp0453-Tp0326 chimeric protein, for example as a means to diagnose syphilis are provided. For example, the methods can include incubating isolated soluble Tp0453 proteins and/or Tp0453-Tp0326 chimeric proteins methods with a serum sample obtained from a human or rabbit subject that may contain Tp0453 or Tp0326 antibodies, under conditions sufficient to permit formation of Tp0453 protein:Tp0453 antibody complexes and Tp0326 protein:Tp0326 antibody complexes. The method can include determining if Tp0453 protein:Tp0453 antibody complexes and Tp0326 protein:Tp0326 antibody complexes were formed and detected. The method can also include determining that the subject has syphilis when Tp0453 protein:Tp0453 antibody complexes or Tp0326 protein:Tp0326 antibody complexes are detected, or that the subject does not have syphilis when Tp0453 protein:Tp0453 antibody complexes or Tp0326 protein:Tp0326 antibody complexes are not detected. In some examples, such methods have a sensitivity of at least 98% (such as at least 99% or 100%) and a specificity of 100%.

### BRIEF DESCRIPTION OF THE DRAWINGS

FIG. 1 is a schematic of the pET28a vector described herein (SEQ ID NO: 4).

FIG. 2 is a chromatogram illustrating the results from the gel filtration chromatography performed on soluble Tp0453 protein.

FIG. 3 is a digital image of a sodium dodecyl sulphate-polyacrylamide gel electrophoresis (SDS-PAGE) performed on fractions of Tp0453 gel filtration.

FIG. 4 is a schematic drawing showing an exemplary Tp0453-Tp0326 protein chimera, which includes a 6-His N-terminal tag, amino acids 32-287 of Tp0453, a glycine-serine linker, and amino acids 22-434 of Tp0326.

FIG. 5 is a digital image of a sodium dodecyl sulphate-polyacrylamide gel electrophoresis (SDS-PAGE) performed on fractions of Tp0453-Tp0326 protein chimera gel filtration.

FIG. 6 is a chromatogram illustrating the results from the gel filtration chromatography performed on soluble Tp0453-Tp0326 chimeric protein.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. In the accompanying sequence listing:

SEQ ID NO: 1 is a nucleic acid coding sequence of a full-length Tp0453.

SEQ ID NO: 2 is a full-length Tp0453 amino acid sequence.

SEQ ID NO: 3 is a Tp0453 fragment (amino acids 32-287 of SEQ ID NO: 2).

SEQ ID NO: 4 is a nucleic acid sequence of pET28a vector.

SEQ ID NOS: 5 and 6 are primer sequences.

SEQ ID NO: 7 is a nucleic acid coding sequence of Tp0326

SEQ ID NO: 8 is a full-length Tp0326 amino acid sequence.

SEQ ID NO: 9 is amino acids 22-434 of the native Tp0326 peptide used in the Tp0453-Tp0326 chimeric construct.

SEQ ID NO: 10 is a nucleic acid coding sequence of an exemplary Tp0453-Tp0326 chimeric construct

SEQ ID NO: 11 is an amino acid sequence of an exemplary Tp0453-Tp0326 chimera, which includes a His tag.

SEQ ID NO: 12 is an amino acid sequence of an exemplary Tp0453-Tp0326 chimera.

SEQ ID NO: 13 is a myc-Tag amino acid sequence that can be used as a purification tag.

SEQ ID NO: 14 is a FLAG-Tag amino acid sequence that can be used as a purification tag.

SEQ ID NO: 15 is an S-Tag amino acid sequence that can be used as a purification tag.

SEQ ID NO: 16 is a glycine-serine repeat amino acid sequence that can be used as a linker.

SEQ ID NO: 17 is an exemplary linker amino acid sequence.

### DETAILED DESCRIPTION OF SEVERAL EMBODIMENTS

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a nucleic acid molecule” includes single or plural nucleic acid molecules and is considered equivalent to the phrase “comprising at least one nucleic acid molecule.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. All GenBank accession numbers and references provided herein are incorporated by reference.

Diagnosis:

The process of identifying a disease by its signs, symptoms and results of various tests, such as syphilis. The conclusion reached through that process is also called “a diagnosis”. Forms of testing commonly performed include blood tests, such as a VDRL test.

Highly Immunoreactive:

An antigen that is more effective at stimulating the immune system than other antigens.

HIV Antigen:

An antigen associated with specific proteins of the HIV virus, which can in some examples be detected in a subject infected with HIV or generates an immune response in an HIV-infected individual and the resulting antibodies can be detected. Exemplary HIV antigens include p24 (e.g., Genbank Accession Nos. AAL98903.1, AAD01386.1 and AAD01376.1), the capsid protein of the HIV virus, gp120 (a glycoprotein encoded by HIV envelope protein), gp41 (a transmembrane glycoprotein encoded by HIV envelope protein), gp160 and gp36. p24 is encoded by the HIV gag gene. HIV-seropositive individuals mount a significant immune response to p24 and thus detection of antibodies to p24 is one basis for determining HIV infection.

Isolated:

A biological component (such as a nucleic acid molecule or protein) that has been substantially separated, produced apart from, or purified away from other biological components in the cell of the organism in which the component naturally occurs, such as other chromosomal and extrachromosomal DNA and RNA, and proteins. Nucleic acid molecules and proteins which have been “isolated” include nucleic acid molecules and proteins purified by standard purification methods. The term also includes nucleic acid molecules and proteins prepared by recombinant expression in a host cell as well as chemically synthesized nucleic acid molecules and peptides. For example, an isolated or “purified” soluble Tp0453 protein or Tp0453-Tp0326 chimeric peptide can be substantially free of other proteins, lipids, carbohydrates or other materials with which it was associated. In one embodiment, a soluble Tp0453 peptide is at least 50%, for example at least 80%, at least 90%, or at least 95% free of other proteins, lipids, carbohydrates or other materials with which it was associated (such as a cell lysate). In one embodiment, a soluble Tp0453-Tp0326 chimeric peptide is at least 50%, for example at least 80%, at least 90%, or at least 95% free of other proteins, lipids, carbohydrates or other materials with which it was associated (such as a cell lysate).

Label:

An agent capable of detection, for example by ELISA, spectrophotometry, flow cytometry, or microscopy. For example, a label can be attached directly or indirectly to a Tp0453 protein or fragment thereof (such as SEQ ID NO: 3), or to a Tp0453-Tp0326 chimeric protein or fragment thereof (such as SEQ ID NO: 11 or 12), thereby permitting detection of the protein. Examples of labels include, but are not limited to, radioactive isotopes, enzyme substrates, co-factors, ligands, chemiluminescent agents, fluorophores, haptens, enzymes, and combinations thereof. Methods for labeling and guidance in the choice of labels appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998). In a particular example, a label is present on a secondary antibody to permit detection of Tp0453 protein/antibody or Tp0453-Tp0326 chimeric protein/antibody complexes, for example to permit diagnosis of syphilis.

Luria Broth:

A nutritionally rich medium containing peptides, vitamins, minerals and trace elements that is used for the growth of bacteria, for example E. coli.

MHA-TP Test:

The microhemmaglutinin antibody confirmatory assay for T. pallidum.

Neurosyphilis.

A site of syphilis infection involving the central nervous system that may occur at any stage of syphilis.

pDEST17:

A gateway expression vector that is used in universal cloning techniques allowing the transfer of DNA between different cloning vectors while maintaining the reading frame, designed by Invitrogen Life Technologies, Cat. No. 11803012.

pET28a:

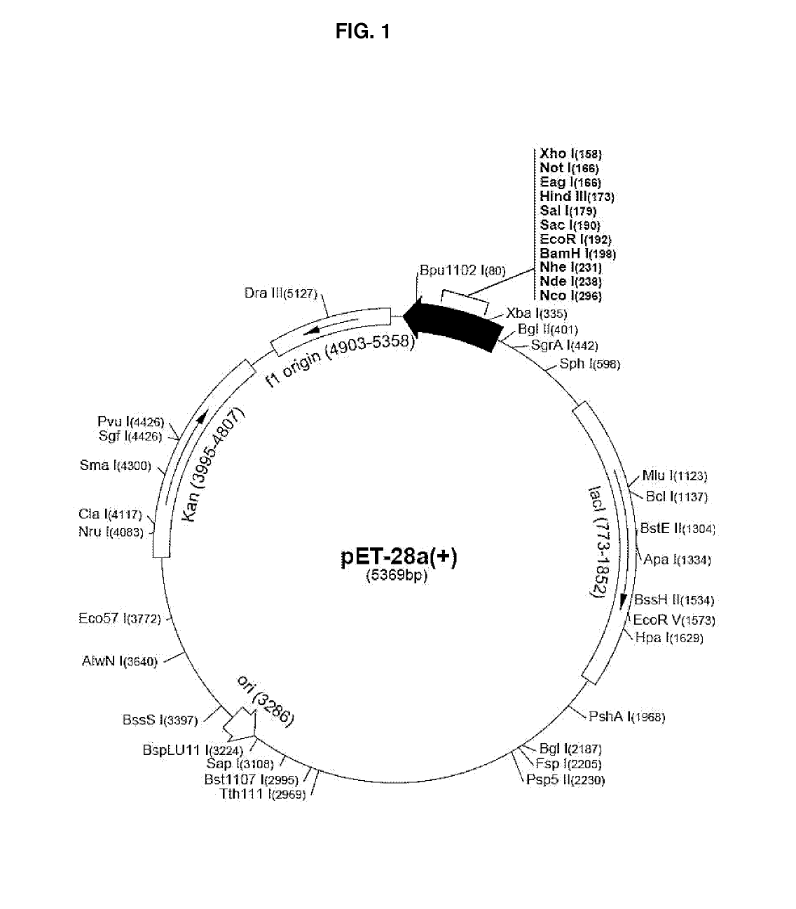

An expression vector shown in FIG. 1 (SEQ ID NO: 4). This vector can be used to express Tp0453 or a fragment thereof (such as SEQ ID NO: 3), for example by inserting a tp0453 coding sequence (such as SEQ ID NO: 1 or nt 94-861 of SEQ ID NO: 1) into the multiple cloning site using the NdeI and XhoI restriction sites. This vector can also be used to express a Tp0453-Tp0326 chimera (such as SEQ ID NO: 11 or 12), for example by inserting a tp0453-tp0326 chimera coding sequence (such as SEQ ID NO: 10 or nt 64-2115 of SEQ ID NO: 10) into the multiple cloning site using the NdeI and XhoI restriction sites.

pET32a:

A vector that is a plasmid designed for cloning and high level expression of peptide or protein sequences, which has been used in research to express insoluble Tp0453.

pRSETc:

An expression vector that is a plasmid, for example that has been used in research to express Tp0453.

RIBI Adjuvant:

An oil-in-water emulsion in which antigens (such as SEQ ID NO: 3, 10 or 11) are mixed with a small volume of metabolizable oil which are then emulsified with saline containing the surfactant Tween 80, it also contains refined mycobacterial products as immunostimulants and bacterial monophsphoryl lipid A. Adjuvants commonly react with immune cells resulting in cytokine induction, enhancing antigen uptake, processing, and presentation.

Sensitivity:

The proportion of positive results that were accurately defined.

Specificity:

The proportion of negative results that were accurately defined.

Sequence Identity/Similarity:

The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site.

BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1554 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 15-nucleotide region that aligns with 20 consecutive nucleotides from an identified sequence as follows contains a region that shares 75 percent sequence identity to that identified sequence (that is, 15÷20*100=75).

For comparisons of amino acid sequences of greater than about 30 amino acids, the Blast 2 sequences function is employed using the default BLOSUM62 matrix set to default parameters, (gap existence cost of 11, and a per residue gap cost of 1). Homologs are typically characterized by possession of at least 30% sequence identity or more counted over the full-length alignment with an amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs use SEG. In addition, a manual alignment can be performed. Proteins with even greater similarity will show increasing percentage identities when assessed by this method, such as at least about 75%, 80%, 85%, 90%, 95%, 98%, or 99% sequence identity with a Tp0453, Tp0326, or Tp0453-Tp0326 chimeric protein, or fragments thereof. Thus in one example, a Tp0453 protein has at least 60%, at least 70%, at least 80%, at least 90%, at least 95%, at least 98%, or at least 99% sequence identity to SEQ ID NO: 2 or 3 and retains the ability to be solubilised using the disclosed methods and can react with antibodies present in sera from an individual with syphilis. In another example, a Tp0326 protein has at least 60%, at least 70%, at least 80%, at least 90%, at least 95%, at least 98%, or at least 99% sequence identity to SEQ ID NO: 8 or 9 and in some examples retains the ability to be solubilised using the disclosed methods and can react with Tp0326 antibodies present in sera from an individual with syphilis. In one example, a Tp0453-Tp0326 chimeric protein has at least 60%, at least 70%, at least 80%, at least 90%, at least 95%, at least 98%, or at least 99% sequence identity to SEQ ID NO: 11 or 12 and retains the ability to be solubilised using the disclosed methods and can react with Tp0453 and Tp0326 antibodies present in sera from an individual with syphilis.

One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. Such homologous nucleic acid sequences can, for example, possess at least about 60%, 70%, 80%, 90%, 95%, 98%, or 99% sequence identity with a Tp0453, Tp0326, or Tp0453-Tp0326 chimera sequence as determined by this method. Thus in one example, a Tp0453 nucleic acid encoding sequence has at least 60%, at least 70%, at least 80%, at least 90%, at least 95%, at least 98%, or at least 99% sequence identity to SEQ ID NO: 1 or nucleotides 94-861 of SEQ ID NO: 1, and encodes a Tp0453 protein or fragment thereof (such as SEQ ID NO: 2 or 3). In one example, a Tp0326 nucleic acid encoding sequence has at least 60%, at least 70%, at least 80%, at least 90%, at least 95%, at least 98%, or at least 99% sequence identity to SEQ ID NO: 7 or nucleotides 877-2115 of SEQ ID NO: 7, and encodes a Tp0326 protein or fragment thereof (such as SEQ ID NO: 8 or 9). Thus in one example, a Tp0453-Tp0326 chimera nucleic acid encoding sequence has at least 60%, at least 70%, at least 80%, at least 90%, at least 95%, at least 98%, or at least 99% sequence identity to SEQ ID NO: 10, and encodes a Tp0453-Tp0326 chimeric protein or fragment thereof (such as SEQ ID NO: 11 or 12). An alternative (and not necessarily cumulative) indication that two nucleic acid sequences are substantially identical is that the polypeptide which the first nucleic acid encodes is immunologically cross reactive with the polypeptide encoded by the second nucleic acid;

Soluble or Solubility:

The ability of a protein, such as Tp0453, Tp0453-Tp0326 chimera, or fragment of Tp0453 or Tp0326, to dissolve in 20 mM Tris and 150 mM NaCl at a pH of 7.5 in the absence of detergents or denaturing agents. It is known that the protein is soluble because at no point in the process is it necessary to add detergents or denaturing agents to ensure that the protein remains in solution, if the protein were to come out of solution it would be visualized in the form of a precipitate. Finally, gel filtration can be used to definitively indicate the solubility of the protein as the protein elutes at the same fraction as a soluble, monomeric protein standard;

Subject:

Living multi-cellular vertebrate organisms, a category that includes human and non-human mammals, for example mammals that get syphilis (such as humans and rabbits).

“Tp0326” (also known as Tp92) refers to a surface antigen of T. pallidum, which has been reported previously to be immunoreactive with serum from syphilis patients (e.g., those infected with T. pallidum). In particular examples, antibodies to Tp0326 are detected in the serum of syphilis patients, in some examples with a sensitivity and specificity of at least 98% and 97% respectively. The term Tp0326 includes a Tp0326 gene, cDNA, mRNA, or protein. Nucleic acid and protein sequences for Tp0326 are publicly available. For example, GenBank Accession No. NC_{—}000919 REGION: 344276 . . . 346837 discloses a Tp0326 nucleic acid sequence, and GenBank Accession No.: AAF61473 discloses a Tp0326 protein sequence, all of which are incorporated by reference as provided by GenBank on Jul. 11, 2011.

In some examples, Tp0326 refers to the full-length protein (e.g., SEQ ID NO: 8) or a fragment thereof (e.g., amino acids 22-434 of the full-length protein, such as the fragment shown in SEQ ID NO: 9). One skilled in the art will appreciate that variations can be made to these sequences, as long as the Tp0326 protein can react with Tp0326 antibodies present in sera from an individual with syphilis. In one example, Tp0326 includes a full-length wild-type (or native) sequence, as well as Tp0326 allelic variants or fragments (such as SEQ ID NO: 9) that can be solubilised using the disclosed methods and can react with antibodies present in a syphilis patient. In certain examples, a Tp0326 protein has at least 80% sequence identity, for example at least 85%, at least 90%, at least 95%, or at least 98% sequence identity to SEQ ID NO: 8 or 9 and can be solubilised using the disclosed methods and can react with Tp0326 antibodies present in sera from an individual with syphilis. In one example, a Tp0326 protein is 413 amino acids (such as SEQ ID NO: 9 or SEQ ID NO: 9 containing 1 to 20 conservative amino acid substitutions), and can further include a tag, such as an N-terminal or C-terminal histidine tag (such as a histidine tag having 3 to 6 histidine residues). In some examples, a Tp0326 nucleic acid sequence has at least 80% sequence identity, for example at least 85%, at least 90%, at least 95%, or at least 98% sequence identity to SEQ ID NO: 7 or nucleotides 877-2115 of SEQ ID NO: 7 and encodes a protein that can react with Tp0326 antibodies present in sera from an individual with syphilis.

Tp0453:

Refers to a protein expressed by T. pallidum which has been reported previously to be immunoreactive with serum from syphilis patients (e.g., those infected with T. pallidum). In particular examples, antibodies to Tp0453 are detected in the serum of syphilis patients, in some examples with a sensitivity and specificity of 100%. The term Tp0453 includes a Tp0453 gene, cDNA, mRNA, or protein. Nucleic acid and protein sequences for Tp0453 are publicly available. For example, GenBank Accession Nos.: AE000520 REGION: 482117 . . . 482980; CP001752.1, 483403 . . . 484209; NC_{—}000919 REGION: 482117 . . . 482980; and CP003115 REGION: 483752 . . . 484615 disclose Tp0453 nucleic acid sequences, and GenBank Accession Nos.: NP_{—}218894, ADD72574, AEZ60777; and AAC65443 disclose Tp0453 protein sequences, all of which are incorporated by reference as provided by GenBank on Feb. 23, 2012.

In some examples, Tp0453 refers to the full-length protein (e.g., SEQ ID NO: 2) or a fragment thereof (e.g., amino acids 32-287 of the full-length protein, such as the fragment shown in SEQ ID NO: 3). One skilled in the art will appreciate that variations can be made to these sequences, as long as the Tp0453 protein can be solubilised using the disclosed methods and can react with sera from an individual with syphilis. In one example, Tp0453 includes a full-length wild-type (or native) sequence, as well as Tp0453 allelic variants or fragments (such as SEQ ID NO: 3) that can be solubilised using the disclosed methods and can react with antibodies present in a syphilis patient. In certain examples, a Tp0453 protein has at least 80% sequence identity, for example at least 85%, 90%, 95%, or 98% sequence identity to SEQ ID NO: 2 or 3 and can be solubilised using the disclosed methods and can react with antibodies present in sera from an individual with syphilis. In one example, a Tp0453 protein is 256 amino acids (such as SEQ ID NO: 3 or SEQ ID NO: 3 containing 1 to 20 conservative amino acid substitutions), and can further include a tag, such as an N-terminal or C-terminal histidine tag (such as a histidine tag having 3 to 6 histidine residues). In some examples, a Tp0453 nucleic acid sequence has at least 80% sequence identity, for example at least 85%, 90%, 95%, or 98% sequence identity to SEQ ID NO: 1 or nucleotides 94-861 of SEQ ID NO: 1 and encodes a protein that can react with antibodies present in sera from an individual with syphilis.

Tp0453-Tp0326 Chimera:

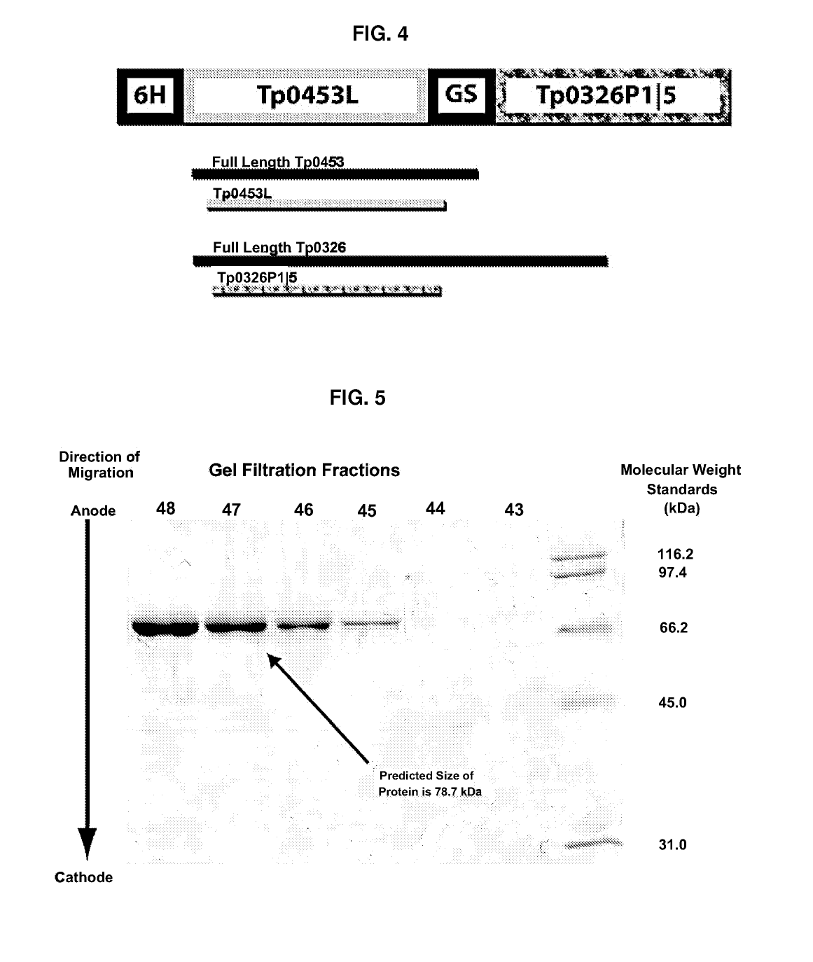

Refers to a fusion protein that includes a soluble Tp0453 portion, and a soluble Tp0326 portion. An exemplary schematic is shown in FIG. 4. Although Tp0453 is shown C-terminal to Tp0326, one will appreciate that this can be reversed. In some examples, the soluble Tp0453 portion comprises or consists of amino acids 32-287 of the full-length Tp0453 protein, such as the fragment shown in SEQ ID NO: 3, and a soluble Tp0326 portion comprises or consists of amino acids 22-434 of the full-length Tp0326 protein, such as the fragment shown in SEQ ID NO: 9. In some examples, the portions are joined by a linker, such as glycine-serine linker (GGGGS; amino acids 1-5 of SEQ ID NO: 16), such as three glycine-serine linkers (SEQ ID NO: 16). The chimera can further include a purification tag, such as a 6-His tag (e.g., on the C-terminus of the chimeric protein). Exemplary chimeras ares shown in SEQ ID NOS: 10 and 11.

VDRL Test:

The Venereal Disease Research Laboratory test which uses anti-cardiolipin, i.e., nontreponemal, antibodies to screen for syphilis infection and that can also be used to assess response to therapy.

### Overview

Syphilis, a disease caused by the bacterium Treponema pallidum subsp. pallidum, remains sensitive to penicillin treatment yet continues to be a global health concern with a burden of at least 25 million cases. The continued prevalence of syphilis is due in part to the shortfalls of current diagnostic tests, which consist of non-specific tests followed by confirmatory tests if a positive result is achieved. Significant limitations associated with these tests include high rates of false positives, poor diagnosis of early and late-stage infections and a reliance on highly specialized expertise and equipment. Although several rapid point-of-care diagnostic tests have been developed, they have shown limited success in detecting early infections.

Described herein is a soluble form of Tp0453, as well as a chimeric protein that includes soluble fragments of both T. pallidum proteins Tp0326 and Tp0453, and methods of their use to detect T. pallidum antibodies in samples from patients with syphilis. Thus, the disclosed soluble form of Tp0453 and Tp045-Tp0326 chimeric proteins can be used in diagnostic tests having greater sensitivity and specificity than current diagnostics.

### Soluble Tp0453 Protein Fragments and Coding Sequences

The present disclosure provides isolated Tp0453 proteins and fragments thereof, such as the full-length protein shown in SEQ ID NO: 2, as well as the fragment shown in SEQ ID NO: 3. One skilled in the art will recognize that such sequences can be altered using routine methods in the art (such as recombinant molecular biology methods), while not significantly decreasing the desired activity of the Tp0453 protein, such as the ability to be solubilised using the disclosed methods and the ability to react with antibodies present in sera from an individual with syphilis. Thus, the present disclosure provides variants of the disclosed Tp0453 sequences, such as a Tp0453 protein sequence having at least 60%, at least 70%, at least 80%, at least 90%, at least 95%, at least 98%, or at least 99% sequence identity to SEQ ID NO: 2 or 3. For example, the peptide sequence shown in SEQ ID NO: 3 of 256 amino acids can be modified by making one or more conservative amino acid substitutions without changing the length of the peptide. Therefore, SEQ ID NO: 3 can be modified by making 1 to 20, 1 to 15, 1 to 12, 1 to 10, or 1 to 5 conservative amino acid substitutions, such as 1, 2, 3, 4, 5, 6, 7, 8, 9, 10, 11, 12, 13, 14, 15, 16, 17, 18, 19, or 20 conservative amino acid substitutions, while retaining the ability to be solubilised using the disclosed methods and react with antibodies present in sera from an individual with syphilis. Examples of conservative substitutions are shown in Table 1:

TABLE 1
Exemplary conservative amino acid substitutions

| Original | Conservative |
|---|---|
| Residue | Substitutions |
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

In some examples, a Tp0453 fragment (such as SEQ ID NO: 3) includes a tag to assist in purification of the protein, such as a C-terminal or N-terminal tag, such as an N-terminal histidine tag. In some examples, the tag is included to aid or permit isolation of the protein, and may or may not be removed before the use of the Tp0453 protein fragment as a diagnostic agent. In some examples, the purification tag is about 3-20 amino acids in length, such as 3 to 10 or 4-6 amino acids. In one example the purification tag is a polyhistidine tag, such as 6-His, which permits nickel purification. Other exemplary purification tags include glutathione-S-transferase (GST), a myc-Tag (EQKLISEED; SEQ ID NO: 13), FLAG-Tag (DYKDDDDK; SEQ ID NO: 14), HA-tag, S-tag (Lys-Glu-Thr-Ala-Ala-Ala-Lys-Phe-Glu-Arg-Gln-His-Met-Asp-Ser; SEQ ID NO: 15), or streptavidin.

Also provided are isolated nucleic acid sequences that encode Tp0453 proteins, such as SEQ ID NO: 1 or nucleotides 94-861 of SEQ ID NO: 1. One skilled in the art will recognize that such sequences can be altered using routine methods in the art (such as recombinant molecular biology methods), while still encoding a Tp0453 protein that retains the ability to be solubilised using the disclosed methods and react with antibodies present in sera from an individual with syphilis. Thus, the present disclosure provides variants of the disclosed Tp0453 nucleic acid sequences, such as a Tp0453 nucleic acid sequence having at least 60%, at least 70%, at least 80%, at least 90%, at least 95%, at least 98%, or at least 99% sequence identity to SEQ ID NO: 1 or nucleotides 94-861 of SEQ ID NO: 1. For example, the sequence shown in SEQ ID NO: 1 or nucleotides 94-861 of SEQ ID NO: 1 can be modified by making one or more nucleotide substitutions, which in some examples will not change the encoded peptide sequence due to the degeneracy of the code, and in other examples will change the sequence of the encoded peptide. For example, a Tp0453 nucleic acid sequence having at least 60%, at least 70%, at least 80%, at least 90%, at least 95%, at least 98%, or at least 99% sequence identity to SEQ ID NO:1 or nucleotides 94-861 of SEQ ID NO: 1, can encode a Tp0453 peptide having 256 amino acids and having at least 60%, at least 70%, at least 80%, at least 90%, at least 95%, at least 98%, or at least 99% sequence identity to SEQ ID NO: 3, while retaining the ability to be solubilised using the disclosed methods and react with antibodies present in sera from an individual with syphilis.

In addition, SEQ ID NO: 1 or nucleotides 94-861 of SEQ ID NO: 1 can be modified by making one or more nucleotide (nt) changes without changing the length of the nucleic acid. Therefore, SEQ ID NO:1 or nt 94-861 of SEQ ID NO: 1 can be modified by making 1 to 100, 1 to 50, 1 to 20, 1 to 15, 1 to 12, 1 to 10, or 1 to 5 nt substitutions, such as 1, 2, 3, 4, 5, 6, 7, 8, 9, 10, 11, 12, 13, 14, 15, 16, 17, 18, 19, 20, 30, 40, 50, 60 70, 80, 90, or 100 nt substitutions, while retaining the ability to encode a protein that can be solubilised using the disclosed methods and react with Tp0453 antibodies present in sera from an individual with syphilis.

Vectors that include a nucleic acid that encodes a Tp0453 protein (such as SEQ ID NO: 3) are also provided. In one example, the vector is pET28a (SEQ ID NO: 4). Thus, provided is a pET28a vector and nucleotides 94-861 of SEQ ID NO: 1.

Also provided are cells that include the vectors and a Tp0453 protein fragment coding sequences (such as nucleotides 94-861 of SEQ ID NO: 1). In one example the cell is a prokaryotic cell, such as E. coli. Other exemplary cells include insect and yeast cells.

### Tp0453-Tp0326 Chimeric Proteins and Coding Sequences

### Protein Sequences

The present disclosure provides isolated Tp0453-Tp0326 chimeric proteins. Such chimeric proteins include a fragment of Tp0453 and a fragment of Tp0326. The Tp0453 and Tp0326 fragments are ideally soluble. An exemplary Tp0453 fragment is shown in SEQ ID NO: 3 (amino acids 32-287 of the native protein), and an exemplary Tp0326 fragment is shown in SEQ ID NO: 9 (amino acids 22-434 of the native protein, which includes all five of polypeptide transport-associated (POTRA) domains).

In particular examples, such a chimeric protein includes the structure X-Y or Y-X. X comprises a soluble fragment of a Tp0453 protein and Y comprises a soluble fragment of a Tp0326 protein. The soluble fragment of a Tp0453 protein can include or consist of the amino acid sequence shown in SEQ ID NO: 3, and the soluble fragment of a Tp0326 protein can include or consist of the amino acid sequence shown in SEQ ID NO: 9. Specific examples of the Tp0453-Tp0326 chimeric protein are shown in SEQ ID NO: 11 and 12.

One skilled in the art will recognize that such sequences can be altered using routine methods in the art (such as recombinant molecular biology methods), while not significantly decreasing the desired activity of the Tp0453-Tp0326 chimeric protein, such as the ability to be solubilised using the disclosed methods and the ability to react with Tp0453 and Tp0326 antibodies present in sera from an individual with syphilis. The immunologic identity of the Tp0453-Tp0326 chimeric protein may be assessed by determining whether it is recognized by Tp0453 and Tp0326 antibodies (such as Tp0453 and Tp0326 antibodies present in a subject having syphilis). Thus, the present disclosure provides variants of the disclosed Tp0453 fragments, Tp0326 fragments, and Tp0453-Tp0326 chimeric sequences.

In one example, the soluble fragment of a Tp0453 protein can have at least 60%, at least 70%, at least 80%, at least 90%, at least 95%, at least 98%, or at least 99% sequence identity to SEQ ID NO: 3. For example, the 256 amino acid sequence shown in SEQ ID NO: 3 can be modified by making one or more conservative amino acid substitutions without changing the length of the peptide. Therefore, SEQ ID NO: 3 can be modified by making 1 to 20, 1 to 15, 1 to 12, 1 to 10, or 1 to 5 conservative amino acid substitutions, such as 1, 2, 3, 4, 5, 6, 7, 8, 9, 10, 11, 12, 13, 14, 15, 16, 17, 18, 19, or 20 conservative amino acid substitutions, while retaining the ability to be solubilised using the disclosed methods and react with Tp0453 antibodies present in sera from an individual with syphilis.

In one example, the soluble fragment of a Tp0326 protein can have at least 60%, at least 70%, at least 80%, at least 90%, at least 95%, at least 98%, or at least 99% sequence identity to SEQ ID NO: 3. For example, the 413 amino acid sequence shown in SEQ ID NO: 9 can be modified by making one or more conservative amino acid substitutions without changing the length of the peptide. Therefore, SEQ ID NO: 9 can be modified by making 1 to 20, 1 to 15, 1 to 12, 1 to 10, or 1 to 5 conservative amino acid substitutions, such as 1, 2, 3, 4, 5, 6, 7, 8, 9, 10, 11, 12, 13, 14, 15, 16, 17, 18, 19, or 20 conservative amino acid substitutions, while retaining the ability to be solubilised using the disclosed methods and react with Tp0326 antibodies present in sera from an individual with syphilis.

In one example, the soluble Tp0453-Tp0326 chimeric protein can have at least 60%, at least 70%, at least 80%, at least 90%, at least 95%, at least 98%, or at least 99% sequence identity to SEQ ID NO: 11 or 12, while retaining the ability to be solubilised using the disclosed methods and react with Tp0453 and Tp0326 antibodies present in sera from an individual with syphilis. In one example, the first 21 aa of SEQ ID NO: 11 are deleted (as they are part of the pET28a vector, resulting in SEQ ID NO: 12). Thus, in one example, the soluble Tp0453-Tp0326 chimeric protein has at least 60%, at least 70%, at least 80%, at least 90%, at least 95%, at least 98%, or at least 99% sequence identity to amino acids 22-705 of SEQ ID NO: 11, while retaining the ability to be solubilised using the disclosed methods and react with Tp0453 and Tp0326 antibodies present in sera from an individual with syphilis. In one example, the Tp0453-Tp0326 chimeric protein comprises or consists of SEQ ID NO: 11 or amino acids 22-705 of SEQ ID NO: 11 (SEQ ID NO: 12).

In addition, the peptide sequence shown in SEQ ID NO: 11 or 12 can be modified by making one or more conservative amino acid substitutions without changing the length of the peptide. Therefore, SEQ ID NO: 11 or 12 can be modified by making 1 to 20, 1 to 15, 1 to 12, 1 to 10, or 1 to 5 conservative amino acid substitutions, such as 1, 2, 3, 4, 5, 6, 7, 8, 9, 10, 11, 12, 13, 14, 15, 16, 17, 18, 19, or 20 conservative amino acid substitutions, while retaining the ability to be solubilised using the disclosed methods and react with Tp0453 and Tp0326 antibodies present in sera from an individual with syphilis. Examples of conservative substitutions are shown in Table 1 above.

In some examples, the Tp0453-Tp0326 chimeric protein is 680-705 aa in length, such as 669 to 690 aa or 684-705 aa, for example 669 aa, 684 aa, 690 aa, or 705 aa. In some examples, the Tp0453-Tp0326 chimeric protein is 684-705 amino acids, wherein the protein comprises amino acids 22-705 of SEQ ID NO: 11, and comprises 0 to 20 or 0 to 10 conservative amino acid substitutions.

The Tp0453-Tp0326 chimeric protein can further include a purification tag (for example at the N-terminus or C-terminus), and/or a linker between the Tp0453 and Tp0326 fragments.

In some examples the chimeric protein has a purification tag, for example at the N- or C-terminus of the chimeric protein. In some examples, the tag is included to aid or permit isolation of the protein, and may or may not be removed before the use of the chimeric protein as a diagnostic agent. In some examples, the purification tag is about 3-20 amino acids in length, such as 3 to 10 or 4-6 amino acids. In one example the purification tag is a polyhistidine tag, such as 6-His, which permits nickel purification. Other exemplary purification tags include glutathione-S-transferase (GST), a myc-Tag (EQKLISEED; SEQ ID NO: 13), FLAG-Tag (DYKDDDDK; SEQ ID NO: 14), HA-tag, S-tag (Lys-Glu-Thr-Ala-Ala-Ala-Lys-Phe-Glu-Arg-Gln-His-Met-Asp-Ser; SEQ ID NO: 15), or streptavidin.

In some examples the chimeric protein has a linker between the Tp0453 and Tp0326 regions. The use of spacer peptides to join two peptide domains is well known in the art; such spacer peptides are typically of between 2 and 25 amino acids in length, and provide a flexible hinge connecting the first peptide sequence to the second peptide. For example, such a linker can be used to separate the two different domains of the chimeric protein and provide flexibility to the chimeric protein. The flexibility allows for proper folding during recombinant expression. The linker also can increase solubility, and may also allow for the formation and availability of epitopes specific to antibodies produced by patients positive for syphilis infection. In some examples, the linker comprises the sequence GGGGS (amino acids 1-5 of SEQ ID NO: 16, such as three of such a sequence (GGGGS x3); SEQ ID NO: 16). In another example, the linker is a proline-threonine linker or a variation of the GGGGS linker that uses arginine (RGRGRGRGRSRGGGS; SEQ ID NO: 17).

### Nucleic Acid Molecules

Isolated nucleic acid molecules encoding the disclosed Tp0453-Tp0326 chimeric proteins are provided herein. In some examples, the nucleotide sequence used for both Tp0453 and Tp0326 is different than that of native T. pallidum, in order to optimize codon usage (for example using codon harmonization).

An exemplary coding sequence for a Tp0453 fragment is shown in nucleotides 64-831 of SEQ ID NO: 10, and an exemplary coding sequence for a Tp0326 fragment is shown in nucleotides 877-2115 of SEQ ID NO: 10. Thus, in some examples, the coding sequence for a soluble fragment of a Tp0453 protein can include or consist of nucleotides 64-831 of SEQ ID NO: 10, and the coding sequence for a soluble fragment of a Tp0326 protein can include or consist of nucleotides 877-2115 of SEQ ID NO: 10. A specific example of the coding sequence for a Tp0453-Tp0326 chimeric protein is shown in SEQ ID NO: 10.

One skilled in the art will recognize that such sequences can be altered using routine methods in the art (such as recombinant molecular biology methods), while not significantly decreasing the desired activity of the Tp0453-Tp0326 chimeric protein, such as the ability to be solubilised using the disclosed methods and the ability to react with Tp0453 and Tp0326 antibodies present in sera from an individual with syphilis.

In one example, the coding sequence for a soluble fragment of a Tp0453 protein can have at least 60%, at least 70%, at least 80%, at least 90%, at least 95%, at least 98%, or at least 99% sequence identity to nucleotides 64-831 of SEQ ID NO: 10. For example, nucleotides 64-831 of SEQ ID NO: 10 can be modified by making one or more nucleotide changes that result in the same amino acid, or result in conservative amino acid substitutions without changing the length of the peptide. Therefore, nucleotides 64-831 of SEQ ID NO: 10 can be modified to result in 1 to 20, 1 to 15, 1 to 12, 1 to 10, or 1 to 5 conservative amino acid substitutions, such as 1, 2, 3, 4, 5, 6, 7, 8, 9, 10, 11, 12, 13, 14, 15, 16, 17, 18, 19, or 20 conservative amino acid substitutions, while retaining the ability to encode a protein that can be solubilised using the disclosed methods and react with Tp0453 antibodies present in sera from an individual with syphilis.

In one example, the coding sequence for a soluble fragment of a Tp0326 protein can have at least 60%, at least 70%, at least 80%, at least 90%, at least 95%, at least 98%, or at least 99% sequence identity to nucleotides 877-2115 of SEQ ID NO: 10. For example, nucleotides 877-2115 of SEQ ID NO: 10 can be modified by making one or more nucleotide changes that result in the same amino acid, or result in conservative amino acid substitutions without changing the length of the peptide. Therefore, nucleotides 877-2115 of SEQ ID NO: 10 can be modified to result in 1 to 20, 1 to 15, 1 to 12, 1 to 10, or 1 to 5 conservative amino acid substitutions, such as 1, 2, 3, 4, 5, 6, 7, 8, 9, 10, 11, 12, 13, 14, 15, 16, 17, 18, 19, or 20 conservative amino acid substitutions, while retaining the ability to encode a protein that can be solubilised using the disclosed methods and react with Tp0326 antibodies present in sera from an individual with syphilis.

In one example, the coding sequence for a soluble Tp0453-Tp0326 chimeric protein can have at least 60%, at least 70%, at least 80%, at least 90%, at least 95%, at least 98%, or at least 99% sequence identity to SEQ ID NO: 10, while retaining the ability to encode a protein that can be solubilised using the disclosed methods and react with Tp0453 and Tp0326 antibodies present in sera from an individual with syphilis. In one example, the first 63 nt are deleted (as they are part of the pET28a vector). Thus, in one example, the soluble Tp0453-Tp0326 chimeric protein has at least 60%, at least 70%, at least 80%, at least 90%, at least 95%, at least 98%, or at least 99% sequence identity to 64-2115 of SEQ ID NO: 10, while retaining the ability to encode a protein that can be solubilised using the disclosed methods and react with Tp0453 and Tp0326 antibodies present in sera from an individual with syphilis. In one example, the Tp0453-Tp0326 chimeric protein coding sequence comprises or consists of SEQ ID NO: 10 or nucleotides 64-2116 of SEQ ID NO: 10.

In addition, the nucleotide sequence shown in SEQ ID NO: 10 can be modified by making one or more nt changes without changing the length of the nucleic acid. Therefore, SEQ ID NO: 10 (or nucleotides 64-2116 of SEQ ID NO: 10) can be modified by making 1 to 100, 1 to 50, 1 to 20, 1 to 15, 1 to 12, 1 to 10, or 1 to 5 nt substitutions, such as 1, 2, 3, 4, 5, 6, 7, 8, 9, 10, 11, 12, 13, 14, 15, 16, 17, 18, 19, 20, 30, 40, 50, 60 70, 80, 90, or 100 nt substitutions, while retaining the ability to encode a protein that can be solubilised using the disclosed methods and react with Tp0453 and Tp0326 antibodies present in sera from an individual with syphilis.

Vectors that include a nucleic acid that encodes a Tp0453-Tp0326 chimeric protein are also provided. In one example, the vector is pET28a (SEQ ID NO: 4). Thus, provided is a pET28a vector and SEQ ID NO: 10 or nucleotides 64-2115 of SEQ ID NO: 10.

Also provided are cells that include the vectors and Tp0453-Tp0326 chimeric protein coding sequences. In one example the cell is a prokaryotic cell, such as E. coli. Other exemplary cells include insect and yeast cells.

### Methods of Making Soluble Tp0453 Protein Fragments and Tp0453-Tp0326 Chimeric Proteins

The present disclosure provides methods for generating soluble recombinant Tp0453 protein and soluble recombinant Tp0453-Tp0326 chimeric proteins. In one aspect, the Tp0453 protein fragment or Tp0453-Tp0326 chimeric protein is highly immunoreactive and therefore suitable for detecting T. pallidum antibodies, for example use in a syphilis diagnostic kit. Due to the high sensitivity and specificity the Tp0453 protein fragment and Tp0453-Tp0326 chimeric protein exhibit, they can provide a more accurate method of diagnostic than any currently known to those skilled in the art. In another aspect, the disclosure provides Tp0453 protein fragments and Tp0453-Tp0326 chimeric proteins in their isolated, soluble form, which can be used for example in diagnostic kits and as an antigen to produce monoclonal antibodies that can be used in for functional and diagnostic investigations.

Methods of producing a soluble Tp0453 protein are provided. The inventors tested numerous vectors and fragments of Tp0453, including amino acids 30-287 of Tp0453 (in pRSETc vector, pDEST17), amino acids 32-287 of Tp0453 (in pET28a, pET32a, pDEST17), amino acids 30-168 of Tp0453 (pRSETc, pET28a, pDEST17), and amino acids 149-287 (pRSETc, pET28a, pDEST17). Of the fragments and vectors tested, the 32-287 amino acid fragment (SEQ ID NO: 3) and the pET28a vector provided the best results. The other vectors and fragments tested did not produce a soluble Tp0453 protein.

Methods of producing a soluble Tp0453-Tp0326 chimeric protein are also provided. The inventors tested numerous fragments for expression of the Tp0453-Tp0326 chimera, including amino acids 32-287, 163-287, and 180-287 of Tp0453 and amino acids 22-183 and 22-434 from Tp0326. Of the fragments and vectors tested, the 32-287 amino acid fragment of Tp0453 and the 22-434 amino acid fragment of Tp0326 provided the best results (that is, a soluble protein that could detect antibodies in serum from syphilis patients). The other fragments tested did not produce a soluble Tp0453-Tp0326 chimeric protein. In separate experiments, chimeric proteins were created using the Multisite-Gateway Pro system (Invitrogen). The regions used were amino acid residues 208-287 for Tp0453, and amino acid residues 25-221, and 271-590 for Tp0326. These regions were joined through recombination (as per the Multisite-Gateway Pro protocol), and the resulting gene sequence was expressed in the expression vector pDEST-17. None of these yielded soluble protein.

In particular examples the methods include expressing a Tp0453 protein or a Tp0453-Tp0326 chimeric protein from a pET28a vector in a cell, and isolating the Tp0453 protein or Tp0453-Tp0326 chimeric protein from the cell. Exemplary cells include prokaryotic cells, such as E. coli, or insect cells or yeast cells. The result is the production of a complete fraction of T. pallidum Tp0453 protein, or Tp0453-Tp0326 chimeric protein, in its soluble form. For example, the expressed Tp0453 protein or Tp0453-Tp0326 chimeric protein can be isolated by lysing the cells, and then isolating the Tp0453 protein or Tp0453-Tp0326 chimeric protein from the lysate. In some examples, the resulting soluble Tp0453 protein or Tp0453-Tp0326 chimeric protein is concentrated before use.

In contrast to methods used in the past, the Tp0453 protein and Tp0453-Tp0326 chimeric protein generated using the disclosed methods, is soluble. For example, the Tp0453 protein or Tp0453-Tp0326 chimeric protein isolated from the cell lysate can be completely dissolved (e.g., does not form a precipitate) in a composition that is composed of 20 mM Tris and 150 mM NaCl at a pH of 7.5, wherein the composition does not contain detergents nor denaturing agents. In one example, the Tp0453 protein or Tp0453-Tp0326 chimeric protein isolated from the cell lysate can be completely dissolved (e.g., does not form a precipitate) in a composition that is composed of 5 to 100 mM Tris and 50 to 500 mM NaCl at a pH of 6 to 8, wherein the composition does not contain detergents nor denaturing agents. The Tp0453 protein or Tp0453-Tp0326 chimeric protein isolated from the cell lysate is soluble because it is not necessary to add detergents or denaturing agents to ensure that the protein remains in solution. In contrast, if the isolated Tp0453 protein or Tp0453-Tp0326 chimeric protein were to come out of solution it would be visualized in the form of a precipitate. Gel filtration can be used to definitively indicate the solubility of the Tp0453 protein or Tp0453-Tp0326 chimeric protein as the isolated Tp0453 protein or Tp0453-Tp0326 chimeric protein elutes at the same fraction as a soluble, monomeric protein standard (such as about 30.8 kDa if SEQ ID NO: 3 is used or about 78.7 kDa if SEQ ID NO: 11 is used). That is, if the Tp0453 protein or Tp0453-Tp0326 chimeric protein were not soluble, it would exhibit particulate matter in the preparation and it would elute at position within the gel filtration trace that correlated with a protein standard that had a significantly higher molecular mass.

Tp0453 and Tp0453-Tp0326 chimeric protein nucleic acid and protein sequences are publicly available and provided herein (see discussion above). In one example, a T. pallidum Tp0453 protein is expressed and isolated, for example a Tp0453 protein having at least 80%, at least 90%, at least 95%, or at least 98% sequence identity to the amino acid sequence shown in SEQ ID NO: 3, and in some examples is 256 amino acids in length. In one example, the Tp0453 protein comprises or consists of the amino acid sequence shown in SEQ ID NO: 3. In one example, a Tp0453-Tp0326 chimeric protein is expressed and isolated, for example a Tp0453-Tp0326 chimeric protein having at least 80%, at least 90%, at least 95%, or at least 98% sequence identity to the amino acid sequence shown in SEQ ID NO: 11 or 12, and in some examples is 705 or 684 amino acids in length. In one example, the Tp0453-Tp0326 chimeric protein comprises or consists of the amino acid sequence shown in SEQ ID NO: 11 or 12. One skilled in the art will appreciate that a Tp0453 protein or Tp0453-Tp0326 chimeric protein can include conservative substitutions to replace one amino acid with another amino acid that is similar in size, hydrophobicity, etc.

Variations in a Tp0453 or Tp0453-Tp0326 chimera sequence (such as a cDNA or genomic sequence or protein sequence) that result in amino acid changes, whether conservative or not, should be minimized in order to preserve the functional and immunologic identity of the encoded protein. Thus, in some examples, a soluble Tp0453 polypeptide (such as SEQ ID NO: 3) includes 1, 2, 5, 10, 12, 15, 20, or 50 conservative substitutions, and in some examples is 256 amino acids but has 1 to 20 conservative substitutions (but no amino acid additions or deletions). In some examples, the soluble Tp0453 polypeptide is 256 amino acids (SEQ ID NO: 3), has 0 to 20 conservative substitutions, and further includes a purification tag. Thus, in some examples, a soluble Tp0453-Tp0326 chimeric polypeptide (such as SEQ ID NO: 11 or 12) includes 1, 2, 5, 10, 12, 15, 20, or 50 conservative substitutions, and in some examples is 705 or 684 amino acids but has 1 to 20 conservative substitutions (but no amino acid additions or deletions). In some examples, the soluble Tp0453-Tp0326 chimeric polypeptide is 684 amino acids (SEQ ID NO: 12), has 0 to 20 conservative substitutions, and further includes a purification tag. The immunologic identity of the Tp0453 protein Tp0453-Tp0326 chimera may be assessed by determining whether it is recognized by an antibody (such as antibodies present in a subject having syphilis).

The expressed Tp0453 protein or Tp0453-Tp0326 chimeric protein can include a tag (for example at the N- or C-terminus) to assist in purifying or isolating the protein from the cell, such as a His-tag, S-tag, glutathione-S-transferase (GST), or streptavidin. In one example, the His-tag includes six histidines. In such examples, the cells can be lysed and the resulting cell lysate exposed to an appropriate immobilized agent, such as Ni²⁺ if the tag is a His-tag (other combinations that can be used include streptavidin and biotin, histidine and silica surfaces modified with nitrilotriacetic acid). The isolated protein can then be eluted (e.g., eluted from Ni^{2±}-coated beads).

### Methods of Making Tp0453 Antibodies

The disclosure also provides methods of producing Tp0453 antibodies using the soluble Tp0453 protein (such as SEQ ID NO: 3) produced by the disclosed methods. Methods of generating antibodies are routine in the art (for example see Harlow and Lane, Using Antibodies: A Laboratory Manual, CSHL, New York, 1999). For example, the method can include generating a soluble Tp0453 protein (such as SEQ ID NO: 3 or a variant having 1-20 conservative amino acid substitutions) using the methods provided herein, administering the soluble Tp0453 protein (for example in the presence of an adjuvant) at an effective dose to a non-human mammal, thereby producing a Tp0453 antibody in the animal. In one example, the antibody is a monoclonal antibody and the non-human mammal is a mouse. In another example, the antibody is a polyclonal antibody and the non-human mammal is a rabbit. Tp0453 antibodies produced by such methods are also provided. In some examples, such antibodies are used to enrich for Tp0453 or to aid in the detection of Tp0453 (e.g., using mass spectrometry).

### Methods of Detecting T. pallidum Antibodies and Diagnosing Syphilis

Methods of diagnosing syphilis using the soluble Tp0453 protein and/or Tp0453-Tp0326 chimeric protein produced by the disclosed methods are disclosed. For example, the disclosed soluble Tp0453 protein (such as SEQ ID NO: 3) and/or Tp0453-Tp0326 chimeric protein (SEQ ID NO: 11 or 12) permit detection of T. pallidum antibodies in a sample. Based on this, methods of diagnosis of syphilis are provided.

In one example, the method includes incubating a disclosed soluble Tp0453 protein and/or Tp0453-Tp0326 chimeric protein with a sample (e.g., serum, plasma, or other blood sample), such as one obtained from a human or rabbit, wherein the sample may contain Tp0453 and/or Tp0326 antibodies. The Tp0453 protein and/or Tp0453-Tp0326 chimeric protein and the serum sample are incubated under conditions sufficient to permit formation of Tp0453 protein:Tp0453 antibody complexes and/or Tp0326 protein:Tp0326 antibody complexes. In some examples, the Tp0453 protein:Tp0453 antibody complexes and/or Tp0326 protein:Tp0326 antibody complexes are quantified. It is then determined whether Tp0453 protein:Tp0453 antibody complexes and/or Tp0326 protein:Tp0326 antibody complexes (e.g., detectable complexes) were generated. The presence of Tp0453 protein:Tp0453 antibody complexes and/or Tp0326 protein:Tp0326 antibody complexes indicates that the subject has syphilis. In contrast, absence of the Tp0453 protein:Tp0453 antibody and/or Tp0326 protein:Tp0326 antibody complexes indicates that the subject does not have syphilis.

Such a method can further include comparing the experimental Tp0453 protein:Tp0453 antibody complexes and/or Tp0326 protein:Tp0326 antibody complexes to a control, such as Tp0453 protein:Tp0453 antibody complexes and/or Tp0326 protein:Tp0326 antibody complexes expected in a subject with syphilis, wherein detecting a value for Tp0453 protein:Tp0453 antibody complexes and/or Tp0326 protein:Tp0326 antibody complexes similar to the control indicates that the subject has syphilis and detecting a value for Tp0453 protein:Tp0453 antibody complexes and/or Tp0326 protein:Tp0326 antibody complexes significantly less than the control indicates that the subject does not have syphilis. For example, the control can be a reference value or range of values expected to be detected in the test/experimental sample if the subject has syphilis, or can be a sample obtained from a sample known to be positive for Tp0453, Tp0326 or syphilis.

In some examples, the method further includes comparing the Tp0453 protein:Tp0453 antibody complexes and/or Tp0326 protein:Tp0326 antibody complexes to a control, wherein detecting a value for Tp0453 protein:Tp0453 antibody complexes and/or Tp0326 protein:Tp0326 antibody complexes similar to the control indicates that the subject does not have syphilis and detecting a value for Tp0453 protein:Tp0453 antibody complexes and/or Tp0326 protein:Tp0326 antibody complexes significantly greater (such as an absorbance value greater than the mean plus two times the standard deviation of the absorbance of the control uninfected sera) than the control indicates that the subject has syphilis. For example, the control can be a reference value or range of values expected to be detected in the test/experimental sample if the subject does not have syphilis, or can be a sample obtained from a sample known to be negative for Tp0453, Tp0326, or syphilis (e.g., when no T. pallidum infection is present).

### Immunoassay Devices Containing Tp0453 and/or Tp0453-Tp0326 Chimeric Proteins

Disclosed herein are immunoassay devices that include a soluble Tp0453 protein (such as SEQ ID NO: 3) and/or a Tp0453-Tp0326 chimeric protein (such as SEQ ID NO: 11 or 12) disclosed herein for determining the presence and/or amount of a T. pallidum antibody (such as Tp0453 and/or Tp0326 antibodies) in a liquid sample, such as human sera. In some examples, a disclosed immunoassay device permits detection of the presence (or absence) of T. pallidum antibodies in a biological sample for diagnosis of syphilis. Such devices can include a solid support, such as a microporous substrate (such as nitrocellulose, nylon, polyvinylidene fluoride (PVDF), polyethersulfone, polycarbonate, polyester, cellulose acetate, mixed cellulose esters, or combinations thereof), as well as ELISA plates and the like.

In some examples, the immunoassay device further includes other antigens that can diagnose other diseases or infections, such as HIV infection. Thus, in some examples, the immunoassay device includes disclosed Tp0453 antigens (such as SEQ ID NO: 3) and/or Tp0453-Tp0326 chimeric proteins (such as SEQ ID NO: 11 or 12), and one or more other diagnostic antigens, such as antigens associated with a sexually transmitted disease (STD) (for example HIV antigens, such as p24, gp41, gp120, gp160, gp36 or combinations thereof; gonorrhoea antigens, such as the L7/L12 ribosomal protein or those in the Gonozyme® assay; Chlamydia trachomatis antigens, such as the LPS antigen or the major outer membrane protein (MOMP); Chancroid (Haemophilus ducreyi) antigens; Granuloma inguinale (Klebsiella granulomatis) antigens; Candidiasis antigens; Hepatitis antigens (such as Hepatitis A, B or C antigens); Herpes simplex for 2 antigens; HPV (Human Papillomavirus) antigens; and the like); lipoidal antigens; tuberculosis (TB) antigens (such as the T-cell antigen ESAT-6 and the CFP10 antigen, as well as those provided in Ireton et al., Clin. Vaccine Immunol. 17:153-47, 2010), or combinations thereof. In one example, the immunoassay device includes disclosed Tp0453 antigens (such as SEQ ID NO: 3) and/or Tp0453-Tp0326 chimeric proteins (such as SEQ ID NO: 11 or 12), and lipoidal antigen or p24 HIV antigen, or both.

Methods of attaching proteins to solid supports are routine in the art. In exemplary devices, a soluble Tp0453 protein (e.g., amino acids 32-287 of the full-length protein, such as the fragment shown in SEQ ID NO: 3) and/or Tp0453-Tp0326 chimeric protein (such as SEQ ID NO: 11 or 12) is immobilized on the device by placing a solution containing the protein(s) onto the device, and allowing the protein to dry on the device. In exemplary devices, the soluble Tp0453 protein and/or Tp0453-Tp0326 chimeric protein is immobilized by a method involving (a) contacting the protein with one or more appropriate antibodies to form an antigen-antibody complex; and (b) applying the antigen-antibody complex to the device (e.g., membrane). In more specific examples, the antigen-antibody complex is immobilized on the device (e.g., membrane) by a method involving (i) immobilizing a Tp0453 antibody and/or Tp0326 antibody on the device (e.g., membrane); (ii) blocking non-specific binding sites on the device (e.g., membrane); (iii) contacting the immobilized antibody with a soluble Tp0453 protein and/or Tp0453-Tp0326 chimeric protein to form an antigen-antibody complex; and (iv) washing the device (e.g., membrane) to remove any unbound soluble Tp0453 and/or Tp0453-Tp0326 chimeric protein.

Immunoassay devices permit the performance of relatively inexpensive, disposable, membrane-based assays for the visual identification of the presence (or absence) of an analyte in a liquid sample. In some examples, such devices permit a point-of-care (POC) assay. Such devices are usually formatted as freestanding dipsticks (e.g., test strips) or as devices having some sort of housing. Typically, an immunoassay device can be used with as little as about 200 μl of liquid sample, and detection of an analyte (such as T. pallidum Abs) in the sample can (but need not) be complete within 2-5 minutes. In clinical assays, the sample may be urine, blood, serum, saliva, or other body fluids. In most instances, no ancillary instrumentation is required to perform such tests, and such devices easily can be used in clinics, laboratories, field locations, and the home even by inexperienced persons.

One principle category of immunoassay is the “sandwich” assay. In general, sandwich immunoassay procedures call for mixing a sample, which may contain an analyte of interest (for example, T. pallidum antibody), with a detector reagent that specifically recognizes the analyte, for example, gold-conjugated Protein A, gold-conjugated Protein G, or gold-conjugated secondary antibody specific for T. pallidum antibody (e.g., anti-human Ab or anti-human Ab(Fc) secondary antibody). The detector reagent is mobile and typically is linked to a label or another signaling reagent, such as dyed latex, or a radioisotope. This mixture is then applied to a chromatographic medium (such as a microporous or bibulous membrane, like nitrocellulose, nylon or PVDF) containing a band or zone of immobilized antigens recognized by the antibody of interest. The chromatographic medium often is in the form of a strip that resembles a dipstick or which can be incorporated into a housing, such as in a lateral flow device or flow-through device. When the complex of the molecule to be assayed and the detector reagent reaches the zone of the immobilized antigens on the chromatographic medium, binding occurs and the detector reagent complex is localized at the zone. This indicates the presence of the molecule to be assayed. This technique can be used to obtain quantitative or semi-quantitative results. Examples of sandwich immunoassays performed on test strips are described in, for example, U.S. Pat. Nos. 4,168,146 and 4,366,241.

In other common forms of membrane-based immunoassays, as typified by some home pregnancy and ovulation detection kits, a test strip (or dipstick) is “dipped” into a sample suspected of containing the subject analyte (such as T. pallidum antibodies). Enzyme-labeled detector reagent is then added, either simultaneously or after an incubation period. The device next is washed and then inserted into a second solution containing a substrate for the enzyme. The enzyme label, if present, interacts with the substrate, causing the formation of colored products, which either deposit as a precipitate onto the solid phase or produce a visible color change in the substrate solution. EP-A 0 125 118 describes such a sandwich type dipstick immunoassay. EP-A 0 282 192 describes a dipstick device for use in competition type assays.

Flow-through type immunoassay devices were designed, in part, to obviate the need for incubation and washing steps associated with dipstick assays. Flow-through immunoassay devices involve a capture reagent (such as a soluble Tp0453 or Tp0453-Tp0326 chimeric protein disclosed herein) bound to a porous membrane or filter to which a liquid sample is added. As the liquid flows through the membrane, target analyte (such as a T. pallidum antibody) binds to the soluble Tp0453 and/or Tp0453-Tp0326 chimeric protein. The addition of sample is followed by addition of detector reagent (such as, gold-conjugated Protein A or gold-conjugate anti-human IgG(Fc)). Alternatively, the detector reagent may be placed on the membrane in a manner that permits the detector to mix with the sample and thereby label the T. palladium antibody. The visual detection of detector reagent provides an indication of the presence of T. pallidum antibody in the sample. Representative flow-through immunoassay devices are described in U.S. Pat. Nos. 4,246,339; 4,277,560; 4,632,901; 4,812,293; 4,920,046; and 5,279,935; and U.S. Pat. Appl. Nos. 20030049857 and 20040241876.

Microfluidic devices can also be used. (e.g., see Chin et al., Nature Med. 17:1015-19, 2011. In one example, the solid support is a microfluidic device, which can be used to detect Tp0453 antibodies (and other targets) in a sample, such as a liquid sample. Such devices are also referred to as “lab-on-a-chip” devices. The development of microfluidics and microfluidic techniques has provided improved chemical and biological research tools, including platforms for performing chemical reactions, combining and separating fluids, diluting samples, and generating gradients (for example, see U.S. Pat. No. 6,645,432). A portable microfluidic device can be transported to almost any location. For microfluidic assays and devices, test samples (such as a liquid sample) can be supplied by an operator, for example using a micropipette. A test sample can be introduced into an inlet of a microfluidic system and the fluid may be drawn through the system by application of a vacuum source to the outlet end of the microfluidic system. Reagents may also be pumped in, for instance by using different syringe pumps filled with the required reagents. After one fluid is pumped into the microfluidic device, a second can be pumped in by disconnecting a line from the first pump and connecting a line from a second pump. Alternatively, valving may be used to switch from one pumped fluid to another. Different pumps can be used for each fluid to avoid cross contamination, for example when two fluids contain components that may react with each other or, when mixed, can affect the results of an assay or reaction. Continuous flow systems can use a series of two different fluids passing serially through a reaction channel. Fluids can be pumped into a channel in serial fashion by switching, through valving, the fluid source that is feeding the tube. The fluids constantly move through the system in sequence and are allowed to react in the channel.

The disclosed immunoassay devices (e.g., microfluidic, flow-through or lateral-flow device) can be used in methods for detecting T. pallidum antibodies and/or diagnosing syphilis in a subject by applying a biological sample (such as blood, serum, skin ulcer exudate, urine, saliva, oral mucosal transudate, or sputum) from a subject to a disclosed device and detecting formation of an immunocomplex among a T. pallidum antibody present in the sample, a soluble Tp0453 protein and/or Tp0453-Tp0326 chimeric protein disclosed herein, and a T. pallidum antibody detector reagent in the detection zone. Detection of formation of the immunocomplex in the capture area or detection zone indicates the presence of a T. pallidum antibody associated with T. pallidum infection and can be used to diagnose syphilis in the subject. In those embodiments in which the device includes a conjugate pad in the path of flow from the sample application area to the capture area, the detected complex includes the mobile or mobilizable detector. In other embodiments in which the detector reagent is applied to the device from an external source, the detected complex includes the externally applied detector. In some embodiments, a detector reagent is labeled Protein A, Fc-specific Protein G, or anti-human antibody.

To detect the formation of an immunocomplex between the T. pallidum antibody in a sample and the immobilized Tp0453 protein (e.g., SEQ ID NO: 3) and/or Tp0453-Tp0326 chimeric protein (e.g., SEQ ID NO: 11 or 12), the T. pallidum antibody capture area can include a single or multiple detection mechanisms and/or conformations. For example, the T. pallidum antibody area may have one or more lines or symbols that display positive conformation of an immunocomplex between the immobilized Tp0453 protein (e.g., SEQ ID NO: 3) and/or Tp0453-Tp0326 chimeric protein and the T. pallidum antibody, for example, as an appearance of color, change in color, change in color intensity within the T. pallidum antibody capture area.

In some embodiments, a disclosed immunoassay device further includes an HIV capture area having either (a) an immobilized HIV antigen capable of being specifically bound by an HIV antibody (which can be detected by a labeled secondary antibody), or (b) an immobilized HIV antibody that specifically binds a mobile HIV antigen. In one example the HIV antigen is one or more of p24, gp41, gp120, gp160, gp36, and similarly antibodies to these proteins can also be used. In these embodiments, a liquid sample applied in the sample application area can flow through or along the membrane to the T. pallidum antibody capture area and to the HIV capture area. Lateral flow devices that include immobilized HIV antigens or antibodies that can be used in combination with the disclosed devices for detecting Tp0453 antibodies are known (e.g., see Nabatiyan et al., JAIDS 53:55-61, 2010; the OraQuick® HIV antibody test; and US Publication No. 2004/0191760). For example, a lateral flow device can include a first region (e.g., first test line containing Tp0453 protein) for detecting Tp0453 and a second region (e.g., second test line containing HIV antigen) for detecting HIV.

In some embodiments, a disclosed immunoassay device further includes a lipoidal capture area having either (a) an immobilized lipoidal antigen capable of being specifically bound by a lipoidal antibody (which can be detected by a labeled secondary antibody), or (b) an immobilized lipoidal antibody that specifically binds a mobile lipoidal antigen. In these embodiments, a liquid sample applied in the sample application area can flow through or along the membrane to the T. pallidum antibody capture area and to the lipoidal capture area.

In some embodiments, a disclosed immunoassay device further includes a TB capture area having either (a) an immobilized TB antigen capable of being specifically bound by an TB antibody (which can be detected by a labeled secondary antibody), or (b) an immobilized TB antibody that specifically binds a mobile TB antigen. In one example the TB antigen is one or more of ESAT-6 and CFP10, and similarly antibodies to these proteins can also be used. In these embodiments, a liquid sample applied in the sample application area can flow through or along the membrane to the T. pallidum antibody capture area and to the TB capture area. For example, a lateral flow device can include a first region (e.g., first test line containing Tp0453 protein and/or Tp0453-Tp0326 chimeric protein) for detecting Tp0453 and/or Tp0326 and a second region (e.g., second test line containing TB antigen) for detecting TB. In some embodiments, a disclosed immunoassay device, such as a lateral flow device, can include a first region (e.g., first test line containing Tp0453 protein) for detecting Tp0453 and a second region (e.g., second test line containing Tp0453-Tp0356 chimeric protein) for detecting Tp0453 and Tp0356.

### Solid Supports

The solid support which forms the foundation of the immunoassay device can be formed from known materials, such as any water immiscible material. In some examples, suitable characteristics of the material that can be used to form the solid support surface include: being amenable to surface activation such that upon activation, the surface of the support is capable of covalently attaching a soluble Tp0453 protein and/or Tp0453-Tp0326 chimeric protein disclosed herein (or an HIV antigen); being chemically inert such that at the areas on the support not occupied by the molecule can bind to T. pallidum antibodies (or HIV antibodies) with high specificity are not amenable to non-specific binding, or when non-specific binding occurs, such materials can be readily removed from the surface without removing the soluble Tp0453 protein and/or Tp0453-Tp0326 chimeric protein (or HIV antigen).

A solid phase can be chosen for its intrinsic ability to attract and immobilize an agent, such as a soluble Tp0453 protein disclosed herein, a Tp0453-Tp0356 chimeric protein disclosed herein, or HIV antigen. Alternatively, the solid phase can possess a factor that has the ability to attract and immobilize a soluble Tp0453 protein, Tp0453-Tp0326 chimeric protein, or HIV antigen. The factor can include a charged substance that is oppositely charged with respect to, for example, the soluble Tp0453 protein and/or Tp0453-Tp0326 chimeric protein itself. In another embodiment, a specific binding member (e.g., Tp0453 antibody, Tp0326 antibody, or HIV antibody) may be immobilized upon the solid phase to immobilize a soluble Tp0453 protein and/or Tp0453-Tp0326 chimeric protein. In this example, therefore, the specific binding member enables the indirect binding of the soluble Tp0453 protein and/or Tp0453-Tp0326 chimeric protein to a solid phase material.

The surface of a solid support may be activated by chemical processes that cause covalent linkage of a soluble Tp0453 protein, Tp0453-Tp0326 chimeric protein, Tp0453 antibodies, or Tp0326 antibodies (or other target antigens or antibodies) to the support. However, any other suitable method may be used for immobilizing an agent (e.g., a protein) to a solid support including, without limitation, ionic interactions, hydrophobic interactions, covalent interactions and the like. The particular forces that result in immobilization of a soluble Tp0453 and/or Tp0453-Tp0326 chimeric protein on a solid phase are not important for the methods and devices described herein.

Numerous and varied solid supports are known to those in the art and include, without limitation, bibulous or microporous membranes (such as, nitrocellulose, nylon or PVDF), the walls of wells of a reaction tray, microtiter plate, test tubes, polystyrene beads, magnetic beads, and microparticles (such as latex particles). Further examples of useful solid supports include: natural polymeric carbohydrates and their synthetically modified, cross-linked or substituted derivatives, such as agar, agarose, cross-linked alginic acid, substituted and cross-linked guar gums, cellulose esters, especially with nitric acid and carboxylic acids, mixed cellulose esters, and cellulose ethers; natural polymers containing nitrogen, such as proteins and derivatives, including cross-linked or modified gelatins; natural hydrocarbon polymers, such as latex and rubber; synthetic polymers which may be prepared with suitably porous structures, such as vinyl polymers, including polyethylene, polypropylene, polystyrene, polyvinylchloride, polyvinylacetate and its partially hydrolyzed derivatives, polyacrylamides, polymethacrylates, copolymers and terpolymers of the above polycondensates, such as polyesters, polyamides, and other polymers, such as polyurethanes or polyepoxides; porous inorganic materials such as sulfates or carbonates of alkaline earth metals and magnesium, including barium sulfate, calcium sulfate, calcium carbonate, silicates of alkali and alkaline earth metals, aluminum and magnesium; and aluminum or silicon oxides or hydrates, such as clays, alumina, talc, kaolin, zeolite, silica gel, or glass (these materials may be used as filters with the above polymeric materials); and mixtures or copolymers of the above classes, such as graft copolymers obtained by initializing polymerization of synthetic polymers on a pre-existing natural polymer.

In one example the solid support is a particle, such as a bead. Such particles can be composed of metal (e.g., gold, silver, platinum), metal compound particles (e.g., zinc oxide, zinc sulfide, copper sulfide, cadmium sulfide), non-metal compound (e.g., silica or a polymer), as well as magnetic particles (e.g., iron oxide, manganese oxide). In some examples the bead is a latex or glass bead. The size of the bead is not critical; exemplary sizes include 5 nm to 5000 nm in diameter. In one example such particles are about 1 μm in diameter.

In another example, the solid support is a bulk material, such as a paper, membrane, porous material, water immiscible gel, water immiscible ionic liquid, water immiscible polymer (such as an organic polymer), and the like. For example, the solid support can comprises a membrane, such as a semi-porous membrane that allows some materials to pass while others are trapped. In one example the membrane comprises nitrocellulose. In a specific example the solid support is part of a lateral flow device that includes a region containing the sensors disclosed herein.

In some embodiments, porous solid supports, such as nitrocellulose, are in the form of sheets or strips, such as those found in a lateral flow device. The thickness of such sheets or strips may vary within wide limits, for example, at least 0.01 mm, at least 0.1 mm, or at least 1 mm, for example from about 0.01 to 5 mm, about 0.01 to 2 mm, about 0.01 to 1 mm, about 0.01 to 0.5 mm, about 0.02 to 0.45 mm, from about 0.05 to 0.3 mm, from about 0.075 to 0.25 mm, from about 0.1 to 0.2 mm, or from about 0.11 to 0.15 mm. The pore size of such sheets or strips may similarly vary within wide limits, for example from about 0.025 to 15 microns, or more specifically from about 0.1 to 3 microns; however, pore size is not intended to be a limiting factor in selection of the solid support. The flow rate of a solid support, where applicable, can also vary within wide limits, for example from about 12.5 to 90 sec/cm (i.e., 50 to 300 sec/4 cm), about 22.5 to 62.5 sec/cm (i.e., 90 to 250 sec/4 cm), about 25 to 62.5 sec/cm (i.e., 100 to 250 sec/4 cm), about 37.5 to 62.5 sec/cm (i.e., 150 to 250 sec/4 cm), or about 50 to 62.5 sec/cm (i.e., 200 to 250 sec/4 cm). In specific embodiments of devices described herein, the flow rate is about 62.5 sec/cm (i.e., 250 sec/4 cm). In other specific embodiments of devices described herein, the flow rate is about 37.5 sec/cm (i.e., 150 sec/4 cm).

In one example, the solid support is composed of an organic polymer. Suitable materials for the solid support include, but are not limited to: polypropylene, polyethylene, polybutylene, polyisobutylene, polybutadiene, polyisoprene, polyvinylpyrrolidine, polytetrafluoroethylene, polyvinylidene difluoroide, polyfluoroethylene-propylene, polyethylenevinyl alcohol, polymethylpentene, polycholorotrifluoroethylene, polysulformes, hydroxylated biaxially oriented polypropylene, aminated biaxially oriented polypropylene, thiolated biaxially oriented polypropylene, etyleneacrylic acid, thylene methacrylic acid, and blends of copolymers thereof). For example, the solid support can be a multi-well ELISA plate.

In yet other examples, the solid support is a material containing, such as a coating containing, any one or more of or a mixture of the ingredients provided herein.

A wide variety of solid supports can be employed in accordance with the present disclosure. Except as otherwise physically constrained, a solid support may be used in any suitable shapes, such as films, sheets, strips, or plates, or it may be coated onto or bonded or laminated to appropriate inert carriers, such as paper, glass, plastic films, or fabrics.

The solid support can be any format to which the molecule specific for the test agent can be affixed, such as microtiter plates, ELISA plates, test tubes, inorganic sheets, dipsticks, lateral flow devices, and the like. One example includes a linear array of molecules specific for the target agent, generally referred to in the art as a dipstick. Another suitable format includes a two-dimensional pattern of discrete cells (such as 4096 squares in a 64 by 64 array). As is appreciated by those skilled in the art, other array formats including, but not limited to slot (rectangular) and circular arrays are equally suitable for use. In one example, the array is formed on a polymer medium, which is a thread, membrane or film. An example of an organic polymer medium is a polypropylene sheet having a thickness on the order of about 1 mil. (0.001 inch) to about 20 mil., although the thickness of the film is not critical and can be varied over a fairly broad range.

In one example the format is a bead, such as a silica bead. In another example the format is a nitrocellulose membrane. In another example the format is filter paper. In yet another example the format is a glass slide. In one example, the solid support is a polypropylene thread. One or more polypropylene threads can be affixed to a plastic dipstick-type device; polypropylene membranes can be affixed to glass slides.

In one example the solid support is a microtiter plate. For example, soluble Tp0453 proteins and/or Tp0453-Tp0326 chimeric proteins can be affixed to the wells of a microtiter plate. The test sample potentially containing T. pallidum antibodies can be placed in the wells of a microtiter plate containing a soluble Tp0453 protein and/or Tp0453-Tp0326 chimeric protein disclosed herein, and the presence of the T. pallidum antibodies detected using the methods provided herein in. The microtiter plate format permits testing of multiple samples simultaneously (together with controls) each in one or more different wells of the same plate; thus, permitting high-throughput analysis of numerous samples.

In some examples, the soluble Tp0453 proteins and/or Tp0453-Tp0326 chimeric proteins are attached to more than one solid support. For example, such proteins can be attached to a bead, which can then be attached to a conjugation pad of a lateral flow device.

Each of the supports and devices discussed herein (e.g., ELISA, lateral flow device) can be, in some embodiments, formatted to detect multiple analytes by the addition of recognition molecules specific for the other analytes of interest (such as HIV and/or lipoidal antibodies). In one embodiment, the device is capable of detecting both anti-lipoidal antibodies (which, for example, are an indicator of active infection) and anti-treponemal antibodies (which, for example, verify reactivity of the non-treponemal test). For example, certain wells of a microtiter plate or regions of a lateral flow device can include proteins specific for the other antibodies associated with syphilis. In one embodiment, the device is capable of detecting both HIV antibodies (which, for example, are an indicator of HIV infection) and Tp0453 and/or Tp0326 antibodies. For example, certain wells of a microtiter plate or regions of a lateral flow device can include HIV antigens associated with HIV infection. Some lateral flow device embodiments can include secondary, tertiary or more capture areas containing recognition molecules specific for the other antibodies associated with syphilis, or antibodies associated with other diseases, such as HIV infection.

### Lateral Flow Devices and Design

In one example, the solid support is a lateral flow device, which can be used to determine the presence and/or amount of one or more T. pallidum antibodies in a fluid sample, for example to diagnose syphilis. Lateral flow devices are commonly known in the art. Briefly, a lateral flow device is an analytical device having as its essence a test strip, through which flows a test sample liquid that is suspected of containing an analyte of interest (such as T. pallidum antibodies). The test liquid and any suspended analyte can flow along the strip to a detection zone in which the analyte (if present) interacts with a capture agent (e.g., soluble Tp0453 proteins and/or Tp0453-Tp0326 chimeric protein) and a detection agent to indicate a presence, absence and/or quantity of the analyte. Lateral flow devices are useful to simplify and automate user sample interface and processing. In some examples, by using such as lateral flow devices, samples can be directly contacted with or applied to the lateral flow device, and no further liquid transfer or mixing is required.

Lateral flow devices are commonly known in the art, and have a wide variety of physical formats. Any physical format that supports and/or houses the basic components of a lateral flow device in the proper function relationship is contemplated by this disclosure. There are a number of commercially available lateral flow type tests and patents disclosing methods for the detection of analytes: U.S. Pat. Nos. 4,313,734; 4,435,504; 4,775,636; 4,703,017; 4,740,468; 4,806,311; 4,806,312; 4,861,711; 4,855,240; 4,857,453; 4,943,522; 4,945,042; 4,496,654; 5,001,049; 5,075,078; 5,126,241; 5,451,504; 5,424,193; 5,712,172; 6,555,390; and 6,368,876; EP 0810436; and WO 92/12428; WO 94/01775; WO 95/16207; and WO 97/06439.

The construction and design of lateral flow devices is very well known in the art, as described, for example, in Millipore Corporation, A Short Guide Developing Immunochromatographic Test Strips, 2nd Edition, pp. 1-40, 1999, available by request at (800) 645-5476; and Schleicher & Schuell, Easy to Work with BioScience, Products and Protocols 2003, pp. 73-98, 2003, 2003, available by request at Schleicher & Schuell BioScience, Inc., 10 Optical Avenue, Keene, N.H. 03431, (603) 352-3810; both of which are incorporated herein by reference.

Devices described herein generally include a strip of absorbent material (such as a microporous membrane), which can be made of different substances each joined to the other in zones, which may be abutted and/or overlapped. In some examples, the absorbent strip can be fixed on a supporting non-interactive material (such as nonwoven polyester), for example, to provide increased rigidity to the strip. Zones within each strip may differentially contain the protein (e.g., soluble Tp0453, Tp0453-Tp0326 chimeric protein, or HIV antigen) required for the detection and/or quantification of the particular antibodies being tested for. Thus these zones can be viewed as functional sectors or functional regions within the test device.

In general, a liquid sample (or a sample suspended in a liquid) is introduced to the strip at the proximal end of the strip, for instance by dipping or spotting. A sample is collected or obtained using methods well known to those skilled in the art. The sample containing (or suspected of containing) the T. pallidum antibodies (or HIV antibodies or lipoidal antibodies) to be detected may be obtained from any biological source. Examples of biological sources include blood serum, blood plasma, chancre exudates, urine, spinal fluid, saliva, oral mucosal transudate, fermentation fluid, lymph fluid, tissue culture fluid and ascites fluid of a human or animal. The sample may be diluted, purified, concentrated, filtered, dissolved, suspended or otherwise manipulated prior to immunoassay to optimize the immunoassay results. The liquid migrates distally through all the functional regions of the strip. The final distribution of the liquid in the individual functional regions depends on the adsorptive capacity and the dimensions of the materials used.

These devices typically include a sample application area and one or more separate target capture areas (conjugation pad) in which an immobilized soluble Tp0453 and/or Tp0453-Tp0326 chimeric protein (or other protein depending on the antibodies to be detected, such as an HIV antibody or lipoidal antibody) is provided. For example, a lateral flow device containing at least two separate target Ab capture areas (such as 2, 3, 4, 5, 6, 7, 8, 9, 10, 11, 12, 13, 14, 15 or more) can be used to detect a plurality of different target T. pallidum Abs or other antibodies associated with syphilis (such as lipoidal antibodies) or other disease (such as HIV infection), in a single sample.

Another common feature of immunoassay devices is a means to detect the formation of a complex between an analyte (such as a T. pallidum antibody) and a capture reagent (such as soluble Tp0453 protein and/or Tp0453-Tp0326 chimeric protein). A detector (also referred to as detector reagent) serves this purpose. A detector may be integrated into an immunoassay device (for example included in a conjugate pad, as described below), or may be applied to the device from an external source. Examples of detector reagents include a labeled Protein A, Protein G, or anti-human antibody, in which the label is one or more of an enzyme, colloidal gold particle, colored latex particle, protein-adsorbed silver particle, protein-adsorbed iron particle, protein-adsorbed copper particle, protein-adsorbed selenium particle, protein-adsorbed sulfur particle, protein-adsorbed tellurium particle, protein-adsorbed carbon particle, or protein-coupled dye sac.

A detector may be a single reagent or a series of reagents that collectively serve the detection purpose. In some instances, a detector reagent is a labeled binding partner specific for the analyte (such as gold-conjugated Protein A, gold-conjugated Fc-specific Protein G, or gold-conjugated anti-human antibody (Fc portion)). In other instances, a detector reagent collectively includes an unlabeled first binding partner specific for the analyte and a labeled second binding partner specific for the first binding partner and so forth. In each instance, a detector reagent specifically detects bound analyte of an analyte-capture reagent complex and, therefore, a detector reagent preferably does not substantially bind to or react with the capture reagent or other components localized in the analyte capture area. Such non-specific binding or reaction of a detector may provide a false positive result. Optionally, a detector reagent can specifically recognize a positive control molecule (such as a non-specific human IgG for a labeled Protein A detector, or a labeled Protein G detector, or a labeled anti-human Ab(Fc)) that is present in a secondary capture area. Preferably, a detector reagent does not substantially bind to or react with an immobilized soluble Tp0453 protein or Tp0453-Tp0326 chimeric protein.

Any liquid (such as a fluid biological sample) applied in the sample application area flows along a path of flow from the sample application area to the detection area, for instance by capillary action. Upon binding of the target antibody in the sample to the protein (e.g., soluble Tp0453, Tp0453-Tp0326 chimera, or HIV antigen), the antibody-protein complex moves to the conjugate pad, where the analyte of interest (e.g., T. pallidum antibodies) can bind (or be bound by) a mobilized or mobilizable detector reagent. For example, a T. pallidum antibody analyte may bind to a gold-conjugated Protein A detector reagent contained in the conjugate pad. The analyte complexed with the detector reagent may subsequently flow to the test result membrane where the complex may further interact with an analyte-specific binding partner (such as soluble Tp0453 protein), which is immobilized. In some examples, a T. pallidum antibody complexed with a detector reagent (such as, gold-conjugated Protein A, gold-conjugated Protein G, or gold-conjugated anti-human Ab) may further bind to unlabeled, immobilized soluble Tp0453 protein and/or Tp0453-Tp0326 chimeric protein. The formation of the immunocomplex between T. pallidum antibody, labeled (e.g., gold-conjugated) detector reagent, and immobilized soluble Tp0453 protein and/or Tp0453-Tp0326 chimeric protein can be detected by the appearance of a visible signal, which results from the accumulation of the label (e.g., gold) in the localized region (such as a proximal). A control line can be included which contain an immobilized, detector-reagent-specific binding partner, which can bind the detector reagent in the presence or absence of the analyte. Such binding at the control line indicates proper performance of the test, even in the absence of the analyte of interest.

In one example where a lateral flow device can detect multiple targets, the device includes a single wicking pad or sample application area, and multiple conjugation pads, membranes and absorption pads (such that each conjugation pad is associated with a particular membrane and absorption pad). For example, each conjugation pad can include a protein specific for a particular target antibody (e.g., p24 protein for HIV antibodies and lipoidal antigen for lipoidal antibodies).

The lateral flow device can include a wicking pad, conjugation pad, membrane, absorption pad, and combinations thereof. Such pads can abut one another or overlap, and can be attached to a backing. Exemplary materials that can be used for the components of a lateral flow device are shown in the table below. However, one of skill in the art will recognize that the particular materials used in a particular lateral flow device will depend on a number of variables, including, for example, the analyte to be detected, the sample volume, the desired flow rate and others, and can routinely select the useful materials accordingly. Exemplary materials are shown in Table 2.

TABLE 2
Exemplary Material for Lateral Flow Devices

| Component | Exemplary Material |
|---|---|
| Wicking Pad | Glass fiber |
| | Woven fibers |
| | Screen |
| | Non-woven fibers |
| | Cellulosic filters |
| | Paper |
| Conjugation Pad | Glass fiber |
| | Polyester |
| | Paper |
| | Surface modified polypropylene |
| Membrane | Nitrocellulose (including pure nitrocellulose and |
| | modified nitrocellulose) |
| | Nitrocellulose direct cast on polyester support |
| | Polyvinylidene fluoride |
| | Nylon |
| Absorption Pad | Cellulosic filters |
| | Paper |

The sample known or suspected of containing T. pallidum antibodies is applied to or contacted with the wicking pad (which is usually at the proximal end of the device), for instance by dipping or spotting. A sample is collected or obtained using methods well known to those skilled in the art. The sample containing the T. pallidum antibodies to be detected may be obtained from any source, such as blood or serum. The sample may be diluted, purified, concentrated, filtered, dissolved, suspended or otherwise manipulated prior to assay to optimize the results. The fluid sample migrates distally through all the functional regions of the strip. The final distribution of the fluid in the individual functional regions depends on the adsorptive capacity and the dimensions of the materials used.

The wicking pad ensures that the sample moves through the device in a controllable manner, such that it flows in a unilateral direction. The wicking pad initially receives the sample, and can serve to remove particulates from the sample. Among the various materials that can be used to construct a sample pad (see Table 2 above), a cellulose sample pad may be beneficial if a large bed volume (e.g., 250 μ/cm²) is a factor in a particular application. In one example, the wicking pad is made of Millipore cellulose fiber sample pads (such as a 10 to 25 mm pad, such as a 15 mm pad). Wicking pads may be treated with one or more release agents, such as buffers, salts, proteins, detergents, and surfactants. Such release agents may be useful, for example, to promote resolubilization of conjugate-pad constituents, and to block non-specific binding sites in other components of a lateral flow device, such as a nitrocellulose membrane. Representative release agents include, for example, trehalose or glucose (1%-5%), PVP or PVA (0.5%-2%), Tween 20 or Triton X-100 (0.1%-1%), casein (1%-2%), SDS (0.02%-5%), and PEG (0.02%-5%).

After contacting the sample to the wicking pad, the sample liquid migrates from bottom to the top because of capillary force. The sample then flows to the conjugation pad, which serves to, among other things, hold the detector reagent. In some embodiments, a detector reagent may be applied externally, for example, from a developer bottle, in which case a lateral flow device need not contain a conjugate pad (see, for example, U.S. Pat. No. 4,740,468). The detector reagent can be immobilized to the conjugation pad by spotting (for example the detector reagent, can be suspended in water or other suitable buffer and spotted onto the conjugation pad and allowed to dry). Detector reagent(s) contained in a conjugate pad is released into solution upon application of the test sample. A conjugate pad may be treated with various substances to influence release of the detector reagent into solution. For example, the conjugate pad may be treated with PVA or PVP (0.5% to 2%) and/or Triton X-100 (0.5%). Other release agents include, without limitation, hydroxypropylmethyl cellulose, SDS, Brij and β-lactose. A mixture of two or more release agents may be used in any given application. In the particular disclosed embodiment, the detector reagent in conjugate pad is labeled Protein A, Protein G, or anti-human IgG(Fc). The conjugation pad can be made of known materials, such as glass fiber, such as one that is 10 to 25 mm, for example 13 mm. When the sample reaches the conjugation pad, T. pallidum Ab-protein complex can bind to the detector reagent in the conjugation pad. This permits detection of the Ab-protein complexes.

In some embodiments of the lateral flow device, a conjugate pad is placed in the path of flow from the sample application area to the T. pallidum antibody capture area. The conjugate pad includes a mobile or mobilizable detector reagent for a T. pallidum antibody, such that flow of liquid through the conjugate pad moves the detector reagent to the T. pallidum antibody capture area. Formation of an immunocomplex among the detector reagent, T. pallidum antibody, and the soluble Tp0453 proteins and/or Tp0453-Tp0326 chimeric proteins provides a visible or otherwise detectable indicator of the presence of the T. pallidum antibody in a biological specimen. In another embodiment, formation of an immunocomplex among the detector reagent, T. pallidum antibody, and the immobilized soluble Tp0453 proteins and/or Tp0453-Tp0326 chimeric proteins in the detection zone provides a visible or otherwise detectable indicator of the presence of the T. pallidum antibody in a biological specimen. In alternative embodiments, the detector reagent is not supplied in a conjugate pad, but is instead applied to the microporous membrane, for example from a developer bottle.

The membrane portion can be made of known materials, such as a HiFlow Plus Cellulose Ester Membrane, such as one that is 10 to 40 mm, for example 25 mm.

The use of an absorbent pad in a lateral flow device is optional. The absorbent pad can act to increase the total volume of sample that enters the device and to draw the sample across the conjugation pad and membrane by capillary action and collect it. This increased volume can be useful, for example, to wash away unbound analyte from the membrane. Any of a variety of materials is useful to prepare an absorbent pad. In some device embodiments, an absorbent pad can be paper (i.e., cellulosic fibers). One of skill in the art may select a paper absorbent pad on the basis of, for example, its thickness, compressibility, manufacturability, and uniformity of bed volume. The volume uptake of an absorbent made may be adjusted by changing the dimensions (usually the length) of an absorbent pad.

Certain embodiments of the lateral flow device also include a lipoidal antibody capture area in the flow path from the sample application area. Such a lipoidal antibody capture area may include, for example, an immobilized antigen having a binding affinity for a mobile lipoidal antibody or an immobilized lipoidal antibody having a binding affinity for a mobile lipoidal antigen. Exemplary lipoidal antigens that can be used are known in the art (for example, see U.S. Pat. Nos. 6,815,173 and 7,888,043; US-2010-0221740-A1; US-2009-0263825-A1). The lateral flow device may also have a mobile or mobilizable detector reagent specific for the lipoidal antibody in the conjugate pad. The detector reagent for the lipoidal antibody may be in the same or a different pad than the detector reagent for the T. pallidum antibody. In particular embodiments, a detector reagent specific for a lipoidal antibody comprises gold-conjugated Protein A, gold-conjugated Fc-specific Protein G, or gold-conjugated anti-human antibody (Fc portion). In other embodiments, a lipoidal capture area can include n lipoidal antibody for capture of a mobile lipoidal antigen present in a liquid sample. In such embodiments, a detector reagent for the mobile lipoidal antigen can be a gold-labeled lipoidal antibody.

### Flow-Through Device Construction and Design

A flow-through device involves a capture reagent (such as a soluble Tp0453 protein and/or Tp0453-Tp0356 chimeric protein disclosed herein) immobilized on a solid support, typically, a membrane (such as, nitrocellulose, nylon, or PVDF). Characteristics of useful membrane have been previously described; however, in a flow-through assay capillary rise is not a particularly important feature of a membrane as the sample moves vertically through the membrane rather than across it as in a lateral flow assay. In a simple representative format, the membrane of a flow-through device is placed in functional or physical contact with an absorbent layer (see, e.g., description of “absorbent pad” above), which acts as a reservoir to draw a liquid sample through the membrane. Optionally, following immobilization of a capture reagent, any remaining protein-binding sites on the membrane can be blocked (either before or concurrent with sample administration) to minimize nonspecific interactions.

In operation of a flow-through device, a liquid sample (such as a bodily fluid sample) is placed in contact with the membrane. Typically, a flow-through device also includes a sample application area (or reservoir) to receive and temporarily retain a liquid sample of a desired volume. The sample passes through the membrane matrix. In this process, an analyte in the sample (such as a T. pallidum antibody) can specifically bind to the immobilized capture reagent (such as a soluble Tp0453 protein and/or Tp0453-Tp0326 chimeric proteins). Where detection of an analyte-capture reagent complex is desired, a detector reagent (such as labeled Protein A, labeled Protein G, or labeled anti-human IgG(Fc)) can be added with the sample or a solution containing a detector reagent can be added subsequent to application of the sample. If an analyte is specifically bound by the capture reagent, a visual representative attributable to the particular detector reagent can be observed on the surface of the membrane. Optional wash steps can be added at any time in the process, for instance, following application of the sample, and/or following application of a detector reagent.

One skilled in the art will appreciate that such a flow-through device can include regions for detection of other targets, such as HIV antigens to detect HIV antibodies and lipoidal antigens to detect lipoidal antibodies.

### Combination Devices

Each of the immunoassay devices discussed above (e.g., dipstick, flow-through device or lateral flow device) can be, in some embodiments, formatted to detect multiple analytes by the addition of secondary, tertiary or more capture areas containing capture reagents specific for other analytes of interest.

In one example, this disclosure contemplates immunoassay devices that concurrently detect lipoidal antibodies and treponemes or T. pallidum antibodies in liquid samples (such as, human serum). Such combination devices further include a lipoidal capture area involving (a) an immobilized lipoidal antigen capable of being specifically bound by lipoidal antibody, or (b) an immobilized lipoidal antibody that specifically binds a mobile lipoidal antigen. As used herein, a “lipoidal antigen” is an antigen containing at least one antigenic determinant that specifically binds lipoidal antibodies. Lipoidal antigens and lipoidal antibodies are polypeptides; thus, when used as capture reagents, these molecules can be directly adhered to a solid support (such as, nitrocellulose, nylon or PVDF). Nonetheless, it is contemplated that lipoidal antigens or lipoidal antibodies can be immobilized (directly or indirectly) on a solid support by any available method.

In one example, this disclosure contemplates immunoassay devices that concurrently detect HIV antibodies and T. pallidum antibodies in a sample (such as, human serum or oral mucosal transudate). Such combination devices further include an HIV capture area involving (a) an immobilized HIV antigen (such as p24) capable of being specifically bound by HIV antibody, or (b) an immobilized HIV antibody that specifically binds a mobile HIV antigen. An “HIV antigen” is an antigen containing at least one antigenic determinant that specifically binds HIV antibodies. HIV antigens and HIV antibodies are polypeptides; thus, when used as capture reagents, these molecules can be directly adhered to a solid support (such as, nitrocellulose, nylon or PVDF). Nonetheless, it is contemplated that HIV antigens or HIV antibodies can be immobilized (directly or indirectly) on a solid support by any available method.

In one example, this disclosure contemplates immunoassay devices that concurrently detect HIV antibodies, lipoidal antibodies, and T. pallidum antibodies in a sample.

A detector reagent can be used to detect the formation of a complex between a lipoidal capture reagent and lipoidal-specific analyte (such as, a lipoidal antigen or a lipoidal antibody), as well as to detect the formation of a complex between an HIV capture reagent and HIV-specific analyte (such as, an HIV antigen or an HIV antibody). In some embodiments, a detector reagent (such as an anti-human Ab(Fc)) can specifically detect a bound treponeme-specific analyte (e.g., a human T. pallidum antibody) and a bound anti-lipoidal antibody analyte (e.g., a human lipoidal antibody) and/or a bound HIV antibody analyte (e.g., a human HIV antibody). In other instances, two or more separate detector reagents for specific detection of a bound treponeme-specific analyte (e.g., T. pallidum antibody or treponemal antigen), a bound anti-lipoidal antibody analyte, and/or a bound HIV antibody analyte, are envisioned.

The operation of an immunoassay device useful for performing concurrent treponemal, non-treponemal and/or HIV tests is substantially similar to devices described elsewhere in this specification. One particular feature of a combination device is that a liquid sample applied to a sample application area is able to contact (e.g., flow to or flow through) each of an anti-lipoidal antibody capture area and/or HIV antibody capture area, and to a treponemal capture area.

### Kits Containing Soluble Tp0453 Protein and/or Tp0453-Tp0326 Chimeric Protein

Isolated soluble Tp0453 proteins (e.g., SEQ ID NO: 3) and/or Tp0453-Tp0326 chimeric proteins (e.g., SEQ ID NO: 11 or 12) can be part of a kit. Such kits can include the soluble Tp0453 protein and/or Tp0453-Tp0326 chimeric protein, as well as other materials, such as instructions for use in detecting T. pallidum antibodies, for example to diagnose syphilis, standard curves or reference values expected if the subject tested has (or does not have syphilis) syphilis. Such kits can also include buffers, labels and the like, including detector reagents. In one example, the soluble Tp0453 protein and/or Tp0453-Tp0326 chimeric protein includes a detectable label, such as a fluorophore. In one example, the kit includes positive and/or negative control samples indicating the presence or absence of syphilis. In some examples, a kit includes one or more the immunoassay devices disclosed herein, for example that also includes a detector reagent.

In some examples, the kit also includes reagents to perform a VDRL assay, a fluorescent treponemal antibody adsorption assay, a microhemagglutination assay for T. pallidum.

In some examples, the kit includes a solid support to which a soluble Tp0453 protein and/or Tp0453-Tp0326 chimeric protein is attached, such as an ELISA plate or lateral flow device or microfluidic device. In some examples, such a solid support also includes other diagnostic antigens or antibodies, such as HIV antigens or antibodies or other STD antigens or antibodies (such as p24), lipoidal antigens or antibodies, tuberculosis (TB) antigens or antibodies, or combinations thereof.

In some examples, the kit includes HIV (or other STD), TB, and/or lipoidal antibodies or antigens, for example attached to a solid support, such as an ELISA plate or lateral flow device. In some examples, the kit includes a solid support to which a soluble Tp0453 protein and/or Tp0453-Tp0326 chimeric protein, and a lipoidal antigen, is attached, such as an ELISA plate or lateral flow device or microfluidic device.

### Example 1

### Generation and Purification of Soluble Tp0453 Fragment

The example describes methods used to generate and purify a soluble form of Tp0453 that has been cleaved of 31 amino acids at the N terminus. The Tp0453 construct included base pairs 94-861 of SEQ ID NO: 1 or amino acids 32-287 of the full length Tp0453 protein (SEQ ID NO: 3). Additionally the native “TAA” stop codon was excluded from the 3′ end of the protein and replaced by a “TGA” stop codon. One skilled in the art will appreciate that conservative amino acid substitutions can be made to SEQ ID NO: 3, without changing the length of the protein, for example by introducing 1 to 20 conservative amino acid substitutions (such as 1 to 5 or 1 to 10 conservative amino acid substitutions).

The Tp0453 fragment corresponding to base pairs 94-861 of SEQ ID NO: 1 was amplified from T. pallidum genomic DNA using polymerase chain reaction techniques and standard protocols. Primers were designed from the 5′ and 3′ ends of the sequence, and incorporated a NdeI restriction site in the 5′ primer and a XhoI restriction site in the 3′ primer. Primer sequences were as follows: 5′ primer (CTA GAC CAT ATG GCA TCA GTA GAT CCG TTG G; SEQ ID NO: 5) and 3′ primer (GTCAG CTC GAG TCA CGA ACT TCC CTT TTT GGA G; SEQ ID NO: 6). In these primers the NdeI site is indicated by underlining and the XhoI site is indicated by bold font.

The amplicon corresponding to tp0453 was ligated first into the cloning vector pJET1 (CloneJet, Fermentas, Ontario, Canada), digested with restriction enzymes NdeI and XhoI followed by ligation into a similarly digested pET28a expression vector (EMD Inc., Mississauga, ON, Canada; see FIG. 1) using standard techniques. The sequence and reading frame of the expression constructs were verified by DNA sequencing with vector-specific primers. The construct was transformed into the E. coli expression strain BL21 Star™ (DE3) (Invitrogen cat. No. C601003).

Expression and purification of the resulting six-histidine-tagged recombinant protein was performed as follows: bacteria were grown in three test tubes containing 5 ml aliquots of Luria Broth (LB)-50 μg/ml Kanamycin for 5 to 7 hours at 37° C. These cultures were then pooled and transferred to four 250 ml Erlenmeyer flasks (1 ml of overnight culture per flask) containing 50 ml each of LB-Kanamycin and incubated overnight for 16-18 hrs at 37° C. The cultures were transferred to six 4 L Erlenmeyer flasks containing 1 L of LB-50 μg/ml Kanamycin (25 ml overnight culture per flask) and incubated at 37° C. for 4-5 hrs until the optical density (OD) at 600 nm reached 0.7-1.0. The temperature was dropped to 25° C. for 0.5-1 hour until an OD of 1.2 was reached, after which the temperature was subsequently dropped to 16° C. After 30 minutes, recombinant expression was induced using a final concentration of 0.4 mM isopropyl-D-thiogalactopyranoside (IPTG) (Invitrogen).

Cultures were grown for an additional 16-18 hrs at 16° C. after induction. Bacteria were harvested by centrifugation, and re-suspended in a solution consisting of 20 mM Tris [pH7.5], 500 mM NaCl, and 20 mM Imidazole in the presence of a protease inhibitor cocktail (EMD Inc.). Cell pellets were frozen in liquid nitrogen and stored at −20° C. After thawing overnight at 4° C., the suspension of cells expressing Tp0453 was lysed in a French press using two passes at 18,000 psi. The resulting lysate was centrifuged at 20,000×g at 4° C. for 45 minutes. The resulting supernatant containing the soluble recombinant protein was removed and subjected to immobilized metal ion affinity chromatography (IMAC) purification, as outlined below.

Soluble histidine-tagged recombinant Tp0453 was isolated using fast protein liquid chromatography (FPLC) and IMAC methodologies. Briefly, using an AKTA Prime Plus FPLC system (GE Healthcare, Baie D'Urfe, QC, Canada), cell-lysis supernatant was applied to a 1 mL H isTrapp FF affinity column (GE Healthcare) containing Ni²⁺-ions immobilized on agarose beads. The column was washed with cold buffer (20 mM Tris pH 7.5, 500 mM NaCl, and 20 mM imidazole), and Ni²⁺-bound proteins were eluted using an imidazole gradient (20-500 mM imidazole). Fractions were monitored with the AKTA prime plus spectrophotometer by measuring the OD at 280 nm, and the purity of the eluted proteins was verified using sodium dodecyl sulfate polyacrylamide gel electrophoresis (SDS-PAGE) and Coomassie brilliant blue staining.

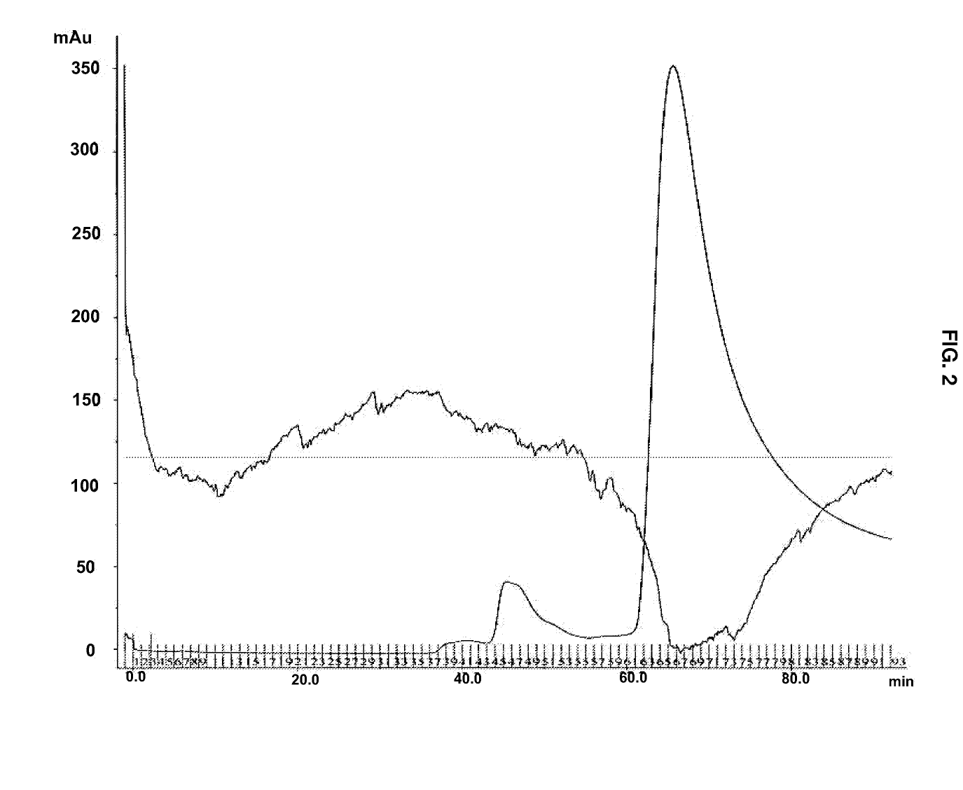

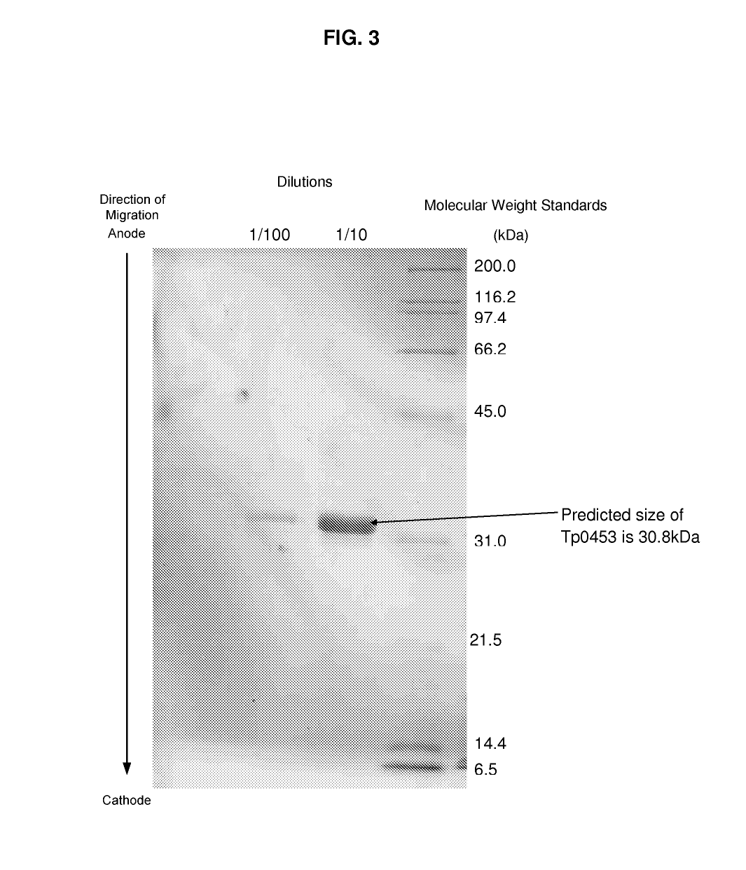

Following Ni²⁺ purification, proteins were concentrated to approximately 2 mL using a 10 kDa molecular mass cut-off centrifugal filter unit (Millipore, Billerica, Mass., USA). Using the AKTA Prime Plus FPLC system, a HiLoad 16/60 Superdex 75 prep grade column (GE Healthcare) was equilibrated with cold buffer (20 mM Tris pH 7.5, 150 mM NaCl), and the concentrated Ni²⁺ purification solution was injected onto the column. Cold buffer (20 mM Tris pH 7.5, 150 mM NaCl) was used to elute the recombinant proteins off the column, and fractions were monitored by measuring the OD at 280 nm. 90 1 ml fractions were collected, the results of the purification are illustrated in FIGS. 2 and 3. Fractions corresponding to the elution peak were examined via SDS-PAGE, pooled, and concentrated (as described above). Recombinant Tp0453 protein concentration was determined using the Thermo Scientific bicinchoninic acid (BCA) protein assay kit (Fischer Scientific Limited, Ottawa, ON, Canada), and pooled protein solutions were flash frozen in liquid nitrogen for 3 minutes and stored at −80° C. Adjustments to the parameters of the procedure may occur in a manner that is known to one skilled in the art.

The chromatogram illustrated in FIG. 2 illustrates soluble Tp0453 protein in its monomeric form eluting in fractions 60-76, these fractions were applied to a 15% SDS-PAGE gel run at 200V for 60 minutes. The gel was run with a Bio-Rad Broad Range Unstained Protein ladder and as shown in FIG. 3, the estimated size of the monomeric pure Tp0453 protein is 30.8 kDa.

The solubility of various Tp0453 fragments was determined in several vectors as shown in Table 3:

TABLE 3
Tp0453 fragments and vectors tested

| Tp0453 | | |
|---|---|---|
| residues | Vectors tested | Result |
| 29-287 | pDEST17 | insoluble protein |
| 30-287 | pRSETc and pDEST17 | insoluble protein |
| 32-287 | pET28a, pET32a, | insoluble protein except with |
| | pDEST17 | pET28a |
| 30-168 | pRSETc, pET28a, | insoluble protein |
| | pDEST17 | |
| 156-287 | pET28a and pET32a | insoluble protein |
| 149-287 | pRSETc, pET28a, | insoluble protein |
| | pDEST17 | |

Of the fragments and vectors tested, the 32-287 amino acid fragment of Tp0453 and the pET28a vector provided soluble protein. The other vectors and fragments tested did not produce a soluble Tp0453 protein.

### Example 2

### Solubility of Tp0453 Fragment

This example describes methods used to measure the solubility of the Tp0453 protein generated in Example 1. One skilled in the art will appreciate that similar methods can be used to measure the solubility of variants of SEQ ID NO: 3, such a peptide having 1 to 20 conservative amino acid substitutions (such as 1 to 5 or 1 to 10 conservative amino acid substitutions).

Initially the recombinant protein was thought to be soluble if it dissolved in 20 mM Tris and 150 mM NaCl at a pH of 7.5 in the absence of detergents or denaturing agents. It is known that the expressed Tp0453 protein was soluble because at no point in the process was it necessary to add detergents or denaturing agents to ensure that the protein remained in solution, if the protein were to become insoluble it would come out of solution it would be visualized in the form of a precipitate.

To definitively indicate the solubility of the Tp0453 protein a gel filtration column was used. Standards were run which elute in a specific predetermined fraction or fractions based on their molecular weight. Because the molecular weight of monomeric Tp0453 is known it can be determined which fraction the soluble form should elute in. In the case that the Tp0453 protein was insoluble or partially soluble, aggregates of the monomer would form, thereby altering the molecular weight and causing it to elute in a different fraction. In this case the recombinant Tp0453 protein produced in Example 1 eluted at the same fraction as the soluble, monomeric protein standard (FIG. 2).

### Example 3

### Generation of Tp0453 Antibodies

This example describes methods that can be used to generate Tp0453 antibodies using the isolated soluble Tp0453 protein generated in Example 1. One skilled in the art will appreciate that similar methods can be used to generate antibodies to variants of SEQ ID NO: 3, such a peptide having 1 to 20 conservative amino acid substitutions (such as 1 to 5 or 1 to 10 conservative amino acid substitutions).

A soluble Tp0453 polypeptide can be used to produce antibodies which are immunoreactive or specifically bind to an epitope of Tp0453. Polyclonal antibodies, antibodies which consist essentially of pooled monoclonal antibodies with different epitopic specificities, as well as distinct monoclonal antibody preparations are included. Antibodies can be raised in small mammals (such as mice and rabbits) using routine methods. Generally, such methods include immunizing the mammal with the soluble Tp0453 protein (such as SEQ ID NO: 3). For example, rabbits can be immunized with 50 μg-200 μg (such as 125 μg) of Tp0453 protein emulsified in an adjuvant (such as RIBI), followed by four boosts using 50 μg-200 μg (such as 125 μg) of protein each.

The preparation of polyclonal antibodies is well known to those skilled in the art. See, for example, Green et al., “Production of Polyclonal Antisera,” in: Immunochemical Protocols, pages 1-5, Manson, ed., Humana Press, 1992; Coligan et al., “Production of Polyclonal Antisera in Rabbits, Rats, Mice and Hamsters,” in: Current Protocols in Immunology, section 2.4.1, 1992.

The preparation of monoclonal antibodies likewise is conventional. See, for example, Kohler & Milstein, Nature 256:495, 1975; Coligan et al., sections 2.5.1-2.6.7; and Harlow et al. in: Antibodies: a Laboratory Manual, page 726, Cold Spring Harbor Pub., 1988. Briefly, monoclonal antibodies can be obtained by injecting mice with a composition containing the soluble Tp0453 protein, verifying the presence of antibody production by removing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan et al., sections 2.7.1-2.7.12 and sections 2.9.1-2.9.3; Barnes et al., Purification of Immunoglobulin G (IgG), in: Methods in Molecular Biology, Vol. 10, pages 79-104, Humana Press, 1992.

Methods of in vitro and in vivo multiplication of monoclonal antibodies are well known to those skilled in the art. Multiplication in vitro may be carried out in suitable culture media such as Dulbecco's Modified Eagle Medium or RPMI 1640 medium, optionally supplemented by a mammalian serum such as fetal calf serum or trace elements and growth-sustaining supplements such as normal mouse peritoneal exudate cells, spleen cells, thymocytes or bone marrow macrophages. Production in vitro provides relatively pure antibody preparations and allows scale-up to yield large amounts of the desired antibodies. Large-scale hybridoma cultivation can be carried out by homogenous suspension culture in an airlift reactor, in a continuous stirrer reactor, or in immobilized or entrapped cell culture. Multiplication in vivo may be carried out by injecting cell clones into mammals histocompatible with the parent cells, such as syngeneic mice, to cause growth of antibody-producing tumors. Optionally, the animals are primed with a hydrocarbon, especially oils such as pristane (tetramethylpentadecane) prior to injection. After one to three weeks, the desired monoclonal antibody is recovered from the body fluid of the animal.

Antibodies can also be derived from a subhuman primate antibody. General techniques for raising therapeutically useful antibodies in baboons can be found, for example, in PCT Publication No. WO 91/11465, 1991; and Losman et al., Int. J. Cancer 46:310, 1990.

Alternatively, an antibody that specifically binds a soluble Tp0453 polypeptide can be derived from a humanized monoclonal antibody. Humanized monoclonal antibodies are produced by transferring mouse complementarity determining regions from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Natl. Acad. Sci. U.S.A. 86:3833, 1989. Techniques for producing humanized monoclonal antibodies are described, for example, by Jones et al., Nature 321:522, 1986; Riechmann et al., Nature 332:323, 1988; Verhoeyen et al., Science 239:1534, 1988; Carter et al., Proc. Natl. Acad. Sci. U.S.A. 89:4285, 1992; Sandhu, Crit. Rev. Biotech. 12:437, 1992; and Singer et al., J. Immunol. 150:2844, 1993.

Antibodies can be derived from human antibody fragments isolated from a combinatorial immunoglobulin library. See, for example, Barbas et al., in: Methods: a Companion to Methods in Enzymology, Vol. 2, page 119, 1991; Winter et al., Ann. Rev. Immunol. 12:433, 1994. Cloning and expression vectors that are useful for producing a human immunoglobulin phage library can be obtained, for example, from STRATAGENE Cloning Systems (La Jolla, Calif.).

In addition, antibodies can be derived from a human monoclonal antibody. Such antibodies are obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13, 1994; Lonberg et al., Nature 368:856, 1994; and Taylor et al., Int. Immunol. 6:579, 1994.

Antibodies include intact molecules as well as fragments thereof, such as Fab, F(ab′)₂, and Fv which are capable of binding the epitopic determinant. These antibody fragments retain some ability to selectively bind with their antigen or receptor and are defined as follows:

(1) Fab, the fragment which contains a monovalent antigen-binding fragment of an antibody molecule, can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;

(4) Fv, defined as a genetically engineered fragment containing the variable region of the light chain and the variable region of the heavy chain expressed as two chains; and

(5) Single chain antibody (SCA), defined as a genetically engineered molecule containing the variable region of the light chain, the variable region of the heavy chain, linked by a suitable polypeptide linker as a genetically fused single chain molecule.

Methods of making these fragments are known in the art (see for example, Harlow and Lane, Antibodies: A Laboratory Manual, Cold Spring Harbor Laboratory, New York, 1988). An epitope is any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics.

Antibody fragments can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see U.S. Pat. No. 4,036,945 and U.S. Pat. No. 4,331,647, and references contained therein; Nisonhoff et al., Arch. Biochem. Biophys. 89:230, 1960; Porter, Biochem. J. 73:119, 1959; Edelman et al., Methods in Enzymology, Vol. 1, page 422, Academic Press, 1967; and Coligan et al. at sections 2.8.1-2.8.10 and 2.10.1-2.10.4).

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

For example, Fv fragments comprise an association of V_{H} and V_{L} chains. This association may be noncovalent (Inbar et al., Proc. Natl. Acad. Sci. U.S.A. 69:2659, 1972). Alternatively, the variable chains can be linked by an intermolecular disulfide bond or cross-linked by chemicals such as glutaraldehyde. See, e.g., Sandhu, supra. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are known in the art (see Whitlow et al., Methods: a Companion to Methods in Enzymology, Vol. 2, page 97, 1991; Bird et al., Science 242:423, 1988; U.S. Pat. No. 4,946,778; Pack et al., Bio/Technology 11:1271, 1993; and Sandhu, supra).

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (Larrick et al., Methods: a Companion to Methods in Enzymology, Vol. 2, page 106, 1991).

Antibodies can be prepared using soluble Tp0453 conjugated to a carrier protein, if desired. Such commonly used carriers which are chemically coupled to the peptide include keyhole limpet hemocyanin (KLH), thyroglobulin, bovine serum albumin (BSA), and tetanus toxoid. The coupled peptide is then used to immunize the animal (e.g., a mouse, a rat, or a rabbit).

Polyclonal or monoclonal antibodies can be further purified, for example, by binding to and elution from a matrix to which the polypeptide or a peptide to which the antibodies were raised is bound. Those of skill in the art will know of various techniques common in the immunology arts for purification and/or concentration of polyclonal antibodies, as well as monoclonal antibodies (see, for example, Coligan et al., Unit 9, Current Protocols in Immunology, Wiley Interscience, 1991).

It is also possible to use the anti-idiotype technology to produce monoclonal antibodies which mimic an epitope. For example, an anti-idiotypic monoclonal antibody made to a first monoclonal antibody will have a binding domain in the hypervariable region that is the “image” of the epitope bound by the first mono-clonal antibody.

Binding affinity for a target Tp0453 antigen is typically measured or determined by standard antibody-antigen assays, such as competitive assays, saturation assays, or immunoassays such as ELISA, EIA, or RIA. Such assays can be used to determine the dissociation constant of the Tp0453 antibody. The phrase “dissociation constant” refers to the affinity of a Tp0453 antibody for a Tp0453 antigen. Specificity of binding between a Tp0453 antibody and a Tp0453 antigen exists if the dissociation constant (K_{D}=1/K, where K is the affinity constant) of the antibody is, for example <1 μM, <100 nM, or <0.1 nM. Tp0453 antibody molecules will typically have a K_{D} in the lower ranges. K_{D}=[Ab−Ag]/[Ab][Ag] where [Ab] is the concentration at equilibrium of the antibody, [Ag] is the concentration at equilibrium of the antigen and [Ab−Ag] is the concentration at equilibrium of the antibody-antigen complex. Typically, the binding interactions between antigen and antibody include reversible noncovalent associations such as electrostatic attraction, Van der Waals forces and hydrogen bonds.

Effector molecules, e.g., detection moieties can be linked to an antibody that specifically binds Tp0453, using any number of means known to those of skill in the art. Exemplary effector molecules include, but not limited to, radiolabels and fluorescent markers. Both covalent and noncovalent attachment means may be used. The procedure for attaching an effector molecule to an antibody varies according to the chemical structure of the effector. Polypeptides typically contain a variety of functional groups; e.g., carboxylic acid (COOH), free amine (—NH₂) or sulfhydryl (—SH) groups, which are available for reaction with a suitable functional group on an antibody to result in the binding of the effector molecule. Alternatively, the antibody is derivatized to expose or attach additional reactive functional groups. The derivatization may involve attachment of any of a number of linker molecules such as those available from Pierce Chemical Company, Rockford, Ill. The linker can be any molecule used to join the antibody to the effector molecule. The linker is capable of forming covalent bonds to both the antibody and to the effector molecule. Suitable linkers are well known to those of skill in the art and include, but are not limited to, straight or branched-chain carbon linkers, heterocyclic carbon linkers, or peptide linkers. Where the antibody and the effector molecule are polypeptides, the linkers may be joined to the constituent amino acids through their side groups (e.g., through a disulfide linkage to cysteine) or to the alpha carbon amino and carboxyl groups of the terminal amino acids.

### Example 4

### Generation and Purification of a Soluble Tp0453-Tp0326 Chimera

The example describes methods used to generate and purify a soluble Tp0453-Tp0326 chimera. The Tp0453-Tp0326 chimera contains amino acids 32-287 of Tp0453 and amino acids 22-434 of Tp0326, linked by a glycine-serine repeat. The sequence of the Tp0453-Tp0326 chimera is shown in SEQ ID NOS: 10 and 11 and is as illustrated in FIG. 4. One skilled in the art will appreciate that conservative amino acid substitutions can be made to SEQ ID NO: 11, without changing the length of the protein, for example by introducing 1 to 20 conservative amino acid substitutions. One skilled in the art will also appreciate that the pET28a sequence that remained at the N-terminus of the chimera can be removed (or the chimera generated without this sequence), resulting in SEQ ID NO 12.

The chimeric protein coding sequence (SEQ ID NO: 11) was created through codon harmonization. The codons used in naturally occurring Tp0453 and Tp0326 (SEQ ID NOS: 1 and 7, respectively) by T. pallidum were changed to reflect comparable frequencies of codon usage seen in E. coli. A glycine-serine repeat (3×GGGGS; SEQ ID NO: 16) was inserted between the Tp0453 and Tp0326, and resulting codon sequences were manually assessed. This linker should not contribute to secondary structure and is flexible, allowing the two regions (Tp0453 and Tp0326) to be separated. The gene containing the 32-287 amino acid fragment of Tp0453 and the 22-434 amino acid fragment of Tp0326, as well as the glycine-serine repeat, was synthesized and inserted into the pIDTSMART-KAN vector.

The plasmid, Tp0453-Tp0326 chimera in pIDTSMART-KAN, was digested with restriction enzymes NdeI and XhoI followed by ligation into a similarly digested pET28a expression vector (Novagen, Gibbstown, N.J., USA; see FIG. 1) using standard techniques. The sequence and reading frame of the expression constructs were verified by DNA sequencing with vector-specific primers. The construct was transformed into the E. coli expression strain BL21 Star™ (DE3) (Invitrogen cat. No. C601003).

Expression and purification of the resulting six-histidine-tagged recombinant protein was performed as follows: bacteria were grown in six test tubes containing 5 ml aliquots of Luria Broth (LB)-50 μg/ml Kanamycin for 5 to 7 hours at 37° C. These cultures were then pooled and transferred to six 250 ml Erlenmeyer flasks (1 ml of overnight culture per flask) containing 50 ml each of LB-Kanamycin and incubated overnight for 16-18 hrs at 37° C. The cultures were then transferred to twelve 4 L Erlenmeyer flasks containing 1.5 L of LB-Kanamycin growth (20 ml overnight culture per flask) and cultured at 37° C. for 4-5 hrs until an OD of 1.4-1.5 was reached. The temperature was then dropped to 25° C. for 0.5-1 hour until an OD of 1.7 was reached. The temperature was then dropped to 16° C. After 30 minutes, recombinant expression was induced using a final concentration of 0.4 mM isopropyl-D-thiogalactopyranoside (Invitrogen).

Cultures were grown for an additional 16-18 hrs at 16° C. after induction. Bacteria were harvested by centrifugation, re-suspended in a solution consisting of 20 mM Tris, 500 mM NaCl, and 20 mM Imidazole, at pH7.5 in the presence of a protease inhibitor cocktail (Catalogue number 539134; Calbiochem, San Diego, Calif., USA). Cells were frozen in liquid nitrogen and stored at −20° C. Cell pellets were thawed overnight at 4° C. and lysis buffer was added to the suspensions of cells expressing the Tp0453-Tp0326 chimera to give a final concentration of 5 mM MgCl₂, 1.6 U/ml DNaseI (Invitrogen), and 5 mg/mL CHAPS. The suspension is incubated at room temperature for 30 minutes. Lysis solution was then sonicated at 10 watts in three 10 second intervals, with 10 second rests between sonications. The resulting solution was then centrifuged at 20,000×g at 4° C. for 45 min, and the supernatant, containing soluble recombinant protein, is recovered.

The soluble recombinant protein preparation was purified using agarose beads with immobilized Ni²⁺-ions according to the 5 mL H isTrapp FF purification protocol (GE Healthcare). Bacterial lysates were centrifuged at 20,000×g at 4° C. for 45 min and the supernatant was applied to Ni^{2±}-agarose beads (GE Healthcare). The column was washed with cold Tris buffer (20 mM Tris pH 7.5, 500 mM NaCl, 20 mM imidazole) and Ni^{2±}-bound proteins were eluted using an imidazole gradient (20-500 mM imidazole).

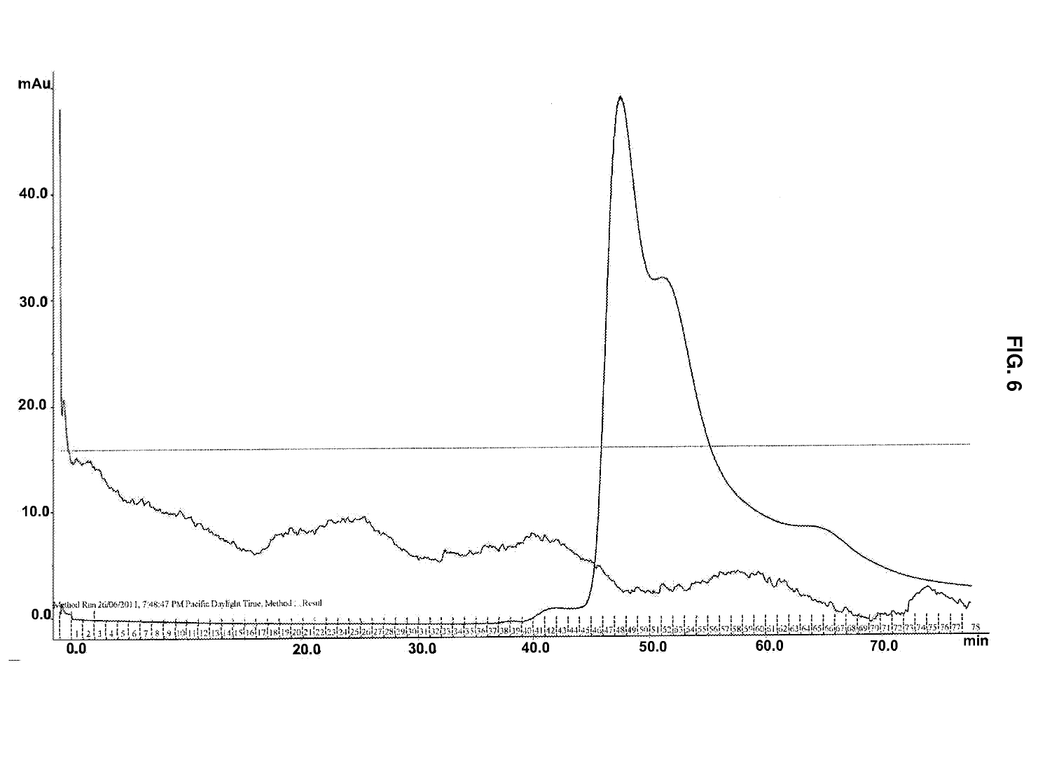

Following Ni purification, the protein was concentrated to approximately 2 mL in a Millipore 10,000 MWCO centrifugal filter unit and loaded onto a HiLoad 16/60 Superdex 75 prep grade column (GE Healthcare). The gel filtration/buffer exchange method template was run on the AKTAprime plus (GE Healthcare) and was performed using 20 mM Tris pH 7.5, 150 mM NaCl as the final buffer. 90 1 ml fractions were collected, the results of the purification are illustrated in FIGS. 5 and 6. Adjustments to the parameters of the procedure may occur in a manner that is known to one skilled in the art.

The chromatogram in FIG. 6 illustrates soluble Tp0453-Tp0326 chimeric protein in its monomeric form eluting in fractions 41-61, these fractions were applied to a 15% SDS-PAGE gel run at 200V for 60 minutes. The gel was run with a Bio-Rad Broad Range Unstained Protein ladder and as shown in FIG. 5, the estimated size of the monomeric pure Tp0453-Tp0326 chimeric protein is 78.7 8 kDa.

The expressed recombinant proteins were flash frozen in liquid nitrogen for 3 minutes and stored at −80° C. The expressed recombinant proteins were quantified using the BCA Protein Assay kit (Pierce, Rockford, Ill., USA)

### Example 5

### ELISA Screening of Sera from Patients Positive or Negative for Syphilis Infection

This example describes methods of using the disclosed soluble Tp0453 proteins and the Tp0453-Tp0326 chimera, to detect T. pallidum antibodies, for example to diagnose syphilis infection.

Enzyme-Linked Immunosorbent Assays.

Frozen protein aliquots were thawed in a water bath at 25° C. Ninety-six well plates (Nunc-Immuno™ MaxiSorp™; Sigma) were incubated at 4° C. overnight with 500 of 6.0 μg/ml recombinant protein solution in phosphate buffered saline (PBS), pH 7.4. Plates were blocked for 2 hours at room temperature with 1×PBS, 4% milk powder. Wells were washed 3 times with wash buffer (1×PBS, 0.05% Tween-20). Human sera was diluted 1:400 in dilution buffer (1×PBS pH 7.4, 4% milk powder, 0.2% Triton X-100), and 50 μl of diluted serum was added to plates in triplicate and incubated for 1 hour at room temperature. Plates were washed with 3 quick washes and 3×10 minute washes. Goat anti-human IgG (Fab specific) peroxidase (Sigma) was diluted to 1:3,000 in dilution buffer (1×PBS pH 7.4, 4% milk powder, 0.2% Triton X-100), and 50 μl was added to each well and incubated for 1 hour at room temperature. Plates were washed with 3 quick washes and 3×10 minute washes. All wash steps, as well as the primary and secondary antibody incubations were done on a rotator at 80 RPM. Plates were developed by adding 100 μl of tetramethylbenzidine-H₂O₂ substrate (Kirkegaard & Perry Laboratories, Gaithersburg, Md., USA) to each well for 30 minutes at room temperature. Absorbance was read at 630 nm.

Statistical Analysis.

Immunoassay cut-off determination was done using serum samples from 24 patients negative for syphilis infection. The samples used were from patients with negative RPR and TP-PA, and differed from the samples used in the final immunological screening. Each sample was run in triplicate in three independent assays. The average absorbance for the negative serum samples over three runs was calculated using the equation:

$${\overline{y}}_{.} = {\sum }_{i = 1}^{n} {\overline{y}}_{i}$$

where n is the number of replicates for each protein, and y represents the average absorbance for each run (over the 24 samples).

The empirical standard error was estimated using the equation:

$$s \hat{e} ( {\overline{y}}_{.} ) = {\{ \frac{{\sum }_{i = 1}^{n} {( {\overline{y}}_{i} - {\overline{y}}_{.} )}^{2}}{n ( n - 1 )} \}}^{1 / 2}$$

Cut-off values using a 98% confidence interval were calculated using the equation:

y̅·±6.965×sê( y̅·)

where 6.965 is the critical value of the T-distribution for a 98% confidence interval, with 2 degrees freedom. The upper-limit of the 98% confidence interval defines the cut-off values for Tp0326 (SEQ ID NO: 9, aa 22-434 of Tp0326), Tp0453 (SEQ ID NO: 3), and the Tp0453-Tp0326 chimera (SEQ ID NO: 11) were 0.138, 0.116, and 0.148 respectively. An equivocal range of greater or less than 10% was calculated, giving ranges of 0.124-0.151, 0.104-0.127 and 0.133-0.163 for Tp0326, Tp0453, and the Tp0453-Tp0326 chimera, respectively. Positive samples were defined as being greater than the equivocal range, and negative samples were defined as being less than the equivocal range. Values falling within the equivocal range were discarded.

Enzyme-Linked Immunosorbent Assay (ELISA) Optimization.

In order to optimize the reagents added to the ELISA analysis, serial dilutions of recombinant protein, patient serum, and the goat anti-human (gamma specific) F(ab′)2 peroxidase secondary antibody were performed. The optimal concentration for Tp0326, Tp0453 and the Tp0453-Tp0326 chimera was found to be 6.0 μg/ml, resulting in a total of 0.3 μg recombinant protein added per well. Three secondary antibodies (two specific for IgG and one specific for IgM) were tested for use in the ELISA, and the goat anti-human IgG (Fab specific) peroxidase was found to be the most sensitive and specific. The ideal dilution for patient serum and goat anti-human peroxidase was found to be 1:400 and 1:3000, respectively. Three independent screenings of serum samples from 38 patients (14 positive and 24 negative) were performed to confirm reproducibility and calculate cut-off values for each of the recombinant proteins.

Serum Panel.

The serum samples were provided by the British Columbia Centre for Disease Control. The characterized positive sera contained 169 samples from patients with confirmed primary (n=70), secondary (n=47), and early latent (n=52) syphilis infection. Positive samples were defined as having both a positive RPR and TP-PA, or in cases where only the RPR or TP-PA test was positive, a positive FTA-ABS test. The serum bank also contained 70 samples from patients negative for syphilis infection including non-reactive (n=11), false positive (positive RPR or TP-PA but a negative FTA-ABS) (n=21), and potentially cross-reactive (n=38) samples. The latter samples included serum samples from patients infected with Borrelia burgdorferi (n=4), Leptospira (n=4), Helicobacter pylori (n=5), Cytomegalovirus (n=5) Epstein-Ban virus (n=5) Hepatitis B virus (n=10) and Hepatitis C virus (n=5). The diagnostic tests used to confirm each cross-reactive sample are listed in Table 4. All cross-reactive samples were screened with the RPR and TP-PA and found to be negative. All serum samples were stored at −20° C. and had undergone only 2 freeze-thaw cycles. All serum samples were blinded prior to screening.

TABLE 4
Diagnostic tests used and manufacturers

| Bacteria/Virus | Test | Manufacturer |
|---|---|---|
| T. pallidum | Rapid plasma reagin | Becton-Dickinson, |
| | | Mississauga, ON, Canada |
| T. pallidum | Treponema pallidum particle | Fujirebio Inc., Malvern, PA, |
| | agglutination assay | USA |
| T. pallidum | Fluorescent treponemal antibody | Zeus Scientific, Branchburg, |
| | absorption assay | NJ, USA |
| B. burgdorferi | Western blot IgG | MarDx Diagnostics, Inc., |
| | | California, USA |
| Leptospira | Panbio ® Leptospira IGM | PanBio, Queensland, Australia |
| H. pylori | H. pylori IgG | Siemens Healthcare, Ontario, |
| | | Canada |
| Cytomegalovirus | VIDAS ® CMV IgG | bioMérieux, Quebec, Canada |
| Epstein-Barr virus | Enzygnost ® Anti-EBV IgG/IgM | Dade Behring/Siemens |
| | | Healthcare, Ontario, Canada |
| Hepatitis B virus | Anti-HBs or Anti-HBc Total | Siemens Healthcare, Ontario, |
| | | Canada |
| Hepatitis C virus | HCV | Siemens Healthcare, Ontario, |
| | | Canada |

Diagnostic Performance of the Recombinant Proteins.

Three proteins, Tp0326 (Cox et al., Mol Microbiol 15:1151-64, 1995; encompasses amino acid residues Q22-N434; SEQ ID NO: 9), Tp0453 (SEQ ID NO: 3), and the Tp0453-Tp0326 chimera (includes the regions used in the tp0326 and tp0453 constructs (amino acids Q22-N434 of Tp0326 and A32-S287 of Tp0453; SEQ ID NO 11; the Tp0453 region was placed N-terminal to the Tp0326 region and a glycine-serine [(GGGGS)₃] linker (SEQ ID NO: 16) was utilized as a flexible spacer between the two regions), were tested against a well-characterized serum panel from syphilis patients to determine the sensitivity of these proteins for diagnosing syphilis infections (Table 5). Positive serum samples (n=169) included patients with confirmed primary (n=70), secondary (n=47), and early latent (n=52) syphilis infection. The overall sensitivity of Tp0326 (SEQ ID NO: 9) was determined to be 86%, with sensitivities of 69%, 98% and 94% for detecting primary, secondary, and early latent infection, respectively. Tp0453 (SEQ ID NO: 3) had an overall sensitivity of 98%, with sensitivities of 96%, 100% and 100% for detecting primary, secondary, and early latent infection, respectively. Finally, the Tp0453-Tp0326 chimera (SEQ ID NO: 11) had an overall sensitivity of 98%, with sensitivities of 94%, 100% and 100% for detecting primary, secondary, and early latent infection, respectively.

TABLE 5
ELISA screening of sera from patients infected with syphilis

| | Positive | Negative | Equivocal | Sensitivity |
|---|---|---|---|---|
| Tp0326 (SEQ ID NO: 9) | | | | |
| Primary | 41 | 18 | 11 | 0.69 |
| Secondary | 45 | 1 | 1 | 0.98 |
| Early Latent | 46 | 3 | 3 | 0.94 |
| All stages | 132 | 22 | 15 | 0.86 |
| Tp0453 (SEQ ID NO: 3) | | | | |
| Primary | 64 | 3 | 3 | 0.96 |
| Secondary | 47 | 0 | 0 | 1.00 |
| Early Latent | 52 | 0 | 0 | 1.00 |
| All stages | 163 | 3 | 3 | 0.98 |
| Chimera (SEQ ID NO: 11) | | | | |
| Primary | 58 | 4 | 8 | 0.94 |
| Secondary | 47 | 0 | 0 | 1.00 |
| Early Latent | 52 | 0 | 0 | 1.00 |
| All stages | 157 | 4 | 8 | 0.98 |

To determine the specificity of the recombinant proteins for accurately detecting only syphilis infection and remaining non-reactive with serum samples from related diseases, potentially cross-reactive conditions or uninfected individuals, the Tp0326, Tp0453 (SEQ ID NO: 3), and Tp0453-Tp0326 chimera recombinant proteins, were tested against serum samples from patients negative for syphilis infection (n=70), including non-reactive (n=11), false positive (positive RPR or TP-PA but a negative FTA-ABS) (n=21), and potential cross-reactive samples (n=38) (Table 6). The overall specificities for Tp0326, Tp0453, and the Tp0453-Tp0326 chimera were 99%, 100%, and 99% respectively. All three proteins exhibited 100% specificity when tested against the non-reactive and biological false positive serum samples. The potentially cross-reactive sera included samples from patients infected with B. burgdorferi (n=4), Leptospira (n=4), H. pylori (n=5), Cytomegalovirus (n=5), Epstein-Barr virus (n=5), hepatitis B virus (n=10), and hepatitis C virus (n=5). Tp0326 exhibited a positive reaction to one serum sample from a patient infected with B. burgdorferi, while the Tp0453-Tp0326 chimera similarly exhibited a positive reaction to one serum sample from a patient infected with the Epstein-Barr virus. Tp0453 showed no reactivity to any of the potentially cross-reactive sera.

TABLE 6
ELISA screening of sera from patients negative for syphilis infection,
including potentially cross-reactive sera.

| | ELISA result | | | |
|---|---|---|---|---|
| | Positive | Negative | Equivocal | Specificity |
| Tp0326 | | | | |
| (SEQ ID NO: 9) | | | | |
| Non-reactive | 0 | 11 | 0 | 1.00 |
| Biological false positives | 0 | 21 | 0 | 1.00 |
| Borrelia burgdorferi | 1 | 3 | 0 | 0.75 |
| Leptospira | 0 | 4 | 0 | 1.00 |
| Helicobacter pylori | 0 | 5 | 0 | 1.00 |
| Cytomegalovirus | 0 | 4 | 1 | 1.00 |
| Epstein-Barr virus | 0 | 5 | 0 | 1.00 |
| Hepatitis B virus | 0 | 10 | 0 | 1.00 |
| Hepatitis C virus | 0 | 5 | 0 | 1.00 |
| All negative samples | 1 | 68 | 1 | 0.99 |
| Tp0453 (SEQ ID NO: 3) | | | | |
| Non-reactive | 0 | 11 | 0 | 1.00 |
| Biological false positives | 0 | 20 | 1 | 1.00 |
| Borrelia burgdorferi | 0 | 4 | 0 | 1.00 |
| Leptospira | 0 | 4 | 0 | 1.00 |
| Helicobacter pylori | 0 | 5 | 0 | 1.00 |
| Cytomegalovirus | 0 | 5 | 0 | 1.00 |
| Epstein-Barr virus | 0 | 5 | 0 | 1.00 |
| Hepatitis B virus | 0 | 10 | 0 | 1.00 |
| Hepatitis C virus | 0 | 5 | 0 | 1.00 |
| All negative samples | 0 | 69 | 1 | 1.00 |
| Tp0453-Tp0326 chimera | | | | |
| (SEQ ID NO: 11) | | | | |
| Non-reactive | 0 | 11 | 0 | 1.00 |
| Biological false positives | 0 | 21 | 0 | 1.00 |
| Borrelia burgdorferi | 0 | 4 | 0 | 1.00 |
| Leptospira | 0 | 4 | 0 | 1.00 |
| Helicobacter pylori | 0 | 5 | 0 | 1.00 |
| Cytomegalovirus | 0 | 5 | 0 | 1.00 |
| Epstein-Barr virus | 1 | 3 | 1 | 0.75 |
| Hepatitis B virus | 0 | 10 | 0 | 1.00 |
| Hepatitis C virus | 0 | 5 | 0 | 1.00 |
| All negative samples | 1 | 68 | 1 | 0.99 |

In summary, Tp0326 exhibits sensitivities and specificities of 86% and 99% respectively. Bioinformatic analysis indicates that Tp0326 is an ortholog of the BamA molecule found throughout Gram-negative bacteria, and the polypeptide transport-associated (POTRA) region used herein is partially conserved (Cameron et al., 2000. J Infect Dis 181:1401-13; Desrosiers et al., 2011. Molecular Microbiology 80:1497-1515, Sánchez-Pulido et al., 2003. Trends Biochem Sci 28:523-6). This may affect its specificity, and explain the reactivity seen in one serum sample from a patient positive for infection with B. burgdorferi. Further, the moderate observed sensitivity of Tp0326 suggests that this protein on its own may not constitute an effective diagnostic candidate.

Tp0453 (SEQ ID NO: 3) was determined to be the best diagnostic candidate in this study, exhibiting a sensitivity of 98% and a specificity of 100%. These results match the previous results of Van Voorhis et al. (J. Clin Microbiol 41:3668-74, 2003), who showed that an insoluble preparation of Tp0453 exhibited 100% sensitivity and 100% specificity upon screening of n=83 serum samples. No serum samples from patients negative for syphilis infection showed reactivity against Tp0453, even those from patients infected with the related spirochetes B. burgdorferi, and Leptospira. The high specificity observed herein may be explained by the sequence of Tp0453, which has no homologues in other bacteria and thus is unique to T. pallidum (Fraser et al., 1998. Science 281:375-88; Hazlett et al., 2005. J Bacteriol 187:6499-508; Mat\check{e}jková et al., 2008. BMC Microbiology 8:76-97). The coding region used in the tp0453 construct is identical across all sequenced strains of T. pallidum, indicating that patients infected with any strain of T. pallidum will likely have antibodies against this protein (Fraser et al., 1998. Science 281:375-88; Mat\check{e}jková et al., 2008. BMC Microbiology 8:76-97).

The strong immunoreactivity exhibited by Tp0326 and Tp0453 indicated that creating a chimera of these two proteins which contains epitopes from both proteins could potentially increase sensitivity. The soluble Tp0453-Tp0326 chimera created shows high sensitivity and specificity (98% and 99% respectively), making this construct another potential candidate for use in syphilis diagnosis.

While this disclosure has been described with an emphasis upon particular embodiments, it will be obvious to those of ordinary skill in the art that variations of the particular embodiments may be used and it is intended that the disclosure may be practiced otherwise than as specifically described herein. Accordingly, this disclosure includes all modifications encompassed within the spirit and scope of the disclosure as defined by the following claims.

1. A method of producing a soluble Tp0453 protein, comprising:
expressing a Tp0453 protein from a pET28a vector in a cell; and
isolating the expressed Tp0453 protein from the cell.

2. (canceled)

3. The method of claim 1, wherein the Tp0453 protein comprises at least 95% sequence identity to the amino acid sequence shown in SEQ ID NO: 3 and is 256 amino acids in length.

4. The method of claim 1, wherein the Tp0453 protein consists of the amino acid sequence shown in SEQ ID NO: 3 and comprises 0 to 15 conservative amino acid substitutions.

5. The method of claim 1, wherein the Tp0453 protein consists of the amino acid sequence shown in SEQ ID NO: 3.

6. The method of claim 1, wherein the Tp0453 protein is expressed in a prokaryotic cell or insect cell.

7. The method of claim 1, wherein the Tp0453 protein further comprises a His-tag, and isolating the expressed Tp0453 protein from the cell comprises:
lysing the cells, thereby generating a cell lysate; and
incubating the cell lysate with immobilized Ni²⁺-ions under conditions to permit binding of the His-tag to the Ni²⁺ ions, thereby isolating the expressed soluble Tp0453 protein.

8. An isolated soluble Tp0453 protein produced by the method of claim 1.

9.-11. (canceled)

12. The isolated protein of claim 10, wherein the protein consists of the amino acid sequence shown in SEQ ID NO: 3.

13. An isolated chimeric protein comprising the structure X-Y or Y-X, wherein:
X comprises a first amino acid sequence comprising the amino acid sequence shown in SEQ ID NO: 3 having 0 to 20 conservative amino acid substitutions; and
Y comprises a second amino acid sequence comprising the amino acid sequence shown in SEQ ID NO: 9 having 0 to 20 conservative amino acid substitutions, wherein the first amino acid sequence is operably linked to the second amino acid, thereby forming the chimeric protein.

14. The isolated chimeric protein of claim 13, further comprising a linker between the first amino acid sequence and the second amino acid sequence, thereby resulting in an isolated chimeric protein comprising the structure X-L-Y or Y-L-X, wherein L is the linker.

15. (canceled)

16. The isolated chimeric protein of claim 13, wherein the isolated chimeric protein comprises at least 95% sequence identity to the amino acid sequence shown in SEQ ID NO: 11 or 12.

17. The isolated chimeric protein of claim 13, wherein the isolated chimeric protein comprises the amino acid sequence shown in SEQ ID NO: 11 or 12.

18. An isolated Tp0453-Tp0326 chimeric protein of 684-705 amino acids, wherein the protein comprises amino acids 22-705 of SEQ ID NO: 11, and comprises 0 to 20 conservative amino acid substitutions.

19. The isolated protein of claim 8, further comprising an N-terminal or a C-terminal purification tag.

20. (canceled)

21. The isolated protein of claim 8, wherein the protein dissolves completely in a solution comprising 20 mM Tris and 150 mM NaCl at a pH of 7.5 and no detergents and no denaturing agents.

22. An isolated nucleic acid molecule encoding the isolated protein of claim 8.

23. An isolated nucleic acid molecule encoding the isolated protein of claim 13, wherein
the nucleic acid molecule encoding the first amino acid sequence comprises nucleotides 64-831 of SEQ ID NO: 10 having 0 to 20 conservative amino acid substitutions; and
the nucleic acid molecule encoding the second amino acid sequence comprises nucleotides 877-2115 of SEQ ID NO: 10 having 0 to 20 conservative amino acid substitutions.

24. The isolated nucleic acid molecule of claim 22, wherein the isolated nucleic acid molecule:
comprises or consists of nucleotides 94-864 of SEQ ID NO: 1, and comprises 1 to 100 nucleotide substitutions;
consists of nucleotides 94-864 of SEQ ID NO: 1;
comprises or consists of nucleotides 64-2115 of SEQ ID NO: 10, and comprises 1 to 100 nucleotide substitutions; or
consists of the nucleic acid sequence of SEQ ID NO: 10.

25. An isolated nucleic acid molecule comprising or consisting of:
a pET28a vector and nucleotides 94-864 of SEQ ID NO: 1; or
a pET28a vector and nucleotides 64-2115 of SEQ ID NO: 10.

26. A vector comprising the isolated nucleic acid of claim 22.

27. The vector of claim 26, wherein the vector comprises pET28a.

28. An isolated cell comprising the vector of claim 26.

29. An immunoassay device, comprising:
a solid support; and
the isolated protein of claim 8 attached to the solid support.

30.-31. (canceled)

32. A kit comprising
the isolated protein of claim 8; and
a detector reagent.

33. A method of diagnosing syphilis, comprising;
incubating the protein of claim 8 with a serum sample obtained from a human or rabbit that may contain Tp0453 and/or Tp0326 antibodies under conditions sufficient to permit formation of Tp0453 protein:Tp0453 antibody complexes and Tp0326 protein:Tp0326 antibody complexes;
determining if Tp0453 protein:Tp0453 antibody complexes and/or Tp0326 protein:Tp0326 antibody complexes were generated;
determining that the subject has syphilis when Tp0453 protein:Tp0453 antibody complexes or Tp0326 protein:Tp0326 antibody complexes are detected or determining that the subject does not have syphilis when of Tp0453 protein:Tp0453 antibody complexes or Tp0326 protein:Tp0326 antibody complexes are not detected.

34.-35. (canceled)

36. The method of claim 33, further comprising:
quantifying the Tp0453 protein:Tp0453 antibody complexes and/or Tp0326 protein:Tp0326 antibody complexes.

37. A method of producing a Tp0453 antibody, comprising administering the soluble Tp0453 protein of claim 8 to a non-human mammal, thereby producing a Tp0453 antibody.

38.-39. (canceled)

40. An antibody produced by the method of claim 37.

41. A method of producing a soluble Tp0453-Tp0326 chimeric protein, comprising:
expressing the protein of claim 13 from a pET28a vector in a cell; and
isolating the expressed protein from the cell.

42. (canceled)

43. An isolated soluble Tp0453-Tp0326 chimeric protein produced by the method of claim 41.


SEQUENCE LISTING (17 sequences)

SEQ ID NO: 1 - DNA, 864 bp, Treponema pallidum
  CDS (1)..(861)
atgataaggcgtagatatcgtggttgtacgcagggagcgtggatagtaagtgttggtatgctatttgcatcgtgcacttcaggggcgtggaaggcatcagtagatccgttgggggttgtgggatctggtgcagatgtgtacctgtatttccctgtagcggggaacgagaatttgatttctcgtattatcgagaaccatgagtcaaaggcagatattaaaaaaatagtggacaggactaccgcggtatacggtgctttttttgcccgatcaaaagagtttcgtttgttcggaagcggttcgtatccatacgcctttactaatttgattttttctcgatccgatggctgggcatctacgaaaacggaacacggaatcacgtactatgaaagtgaacatacggacgtttcgattcctgcgccgcatttttcctgtgtgatttttggttcctccaaaagggagcggatgagcaaaatgctgtctcggctcgttaaccccgatcgaccgcagttaccgcctcgctttgaaaaagaatgtacgtcggaaggtacgagccagactgttgcactctatataaaaaacgggggacactttattaccaaactgttgaattttccgcagcttaatttaccacttggggcaatggaactgtacttgaccgcgcggaggaatgagtatctttacacgttgagcttgcagctggggaatgcaaagataaattttcccatacagtttttaatctctcgtgtgcttaacgcgcacattcacgtggagggggacaggttaattattgaagacggcacaatttctgctgagcgtcttgcgtcggtgatctcgtcgttgtactccaaaaagggaagttcgtaa

SEQ ID NO: 2 - protein, 287 aa, Treponema pallidum
MIRRRYRGCTQGAWIVSVGMLFASCTSGAWKASVDPLGVVGSGADVYLYFPVAGNENLISRIIENHESKADIKKIVDRTTAVYGAFFARSKEFRLFGSGSYPYAFTNLIFSRSDGWASTKTEHGITYYESEHTDVSIPAPHFSCVIFGSSKRERMSKMLSRLVNPDRPQLPPRFEKECTSEGTSQTVALYIKNGGHFITKLLNFPQLNLPLGAMELYLTARRNEYLYTLSLQLGNAKINFPIQFLISRVLNAHIHVEGDRLIIEDGTISAERLASVISSLYSKKGSS

SEQ ID NO: 3 - protein, 256 aa, Artificial Sequence
  Tp0453 fragment
ASVDPLGVVGSGADVYLYFPVAGNENLISRIIENHESKADIKKIVDRTTAVYGAFFARSKEFRLFGSGSYPYAFTNLIFSRSDGWASTKTEHGITYYESEHTDVSIPAPHFSCVIFGSSKRERMSKMLSRLVNPDRPQLPPRFEKECTSEGTSQTVALYIKNGGHFITKLLNFPQLNLPLGAMELYLTARRNEYLYTLSLQLGNAKINFPIQFLISRVLNAHIHVEGDRLIIEDGTISAERLASVISSLYSKKGSS

SEQ ID NO: 4 - DNA, 5369 bp, Artificial Sequence
  pET28a vector
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatccgcgacccatttgctgtccaccagtcatgctagccatatggctgccgcgcggcaccaggccgctgctgtgatgatgatgatgatggctgctgcccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 5 - DNA, 31 bp, Artificial Sequence
  5′-primer
ctagaccatatggcatcagtagatccgttgg

SEQ ID NO: 6 - DNA, 33 bp, Artificial Sequence
  3′-primer
gtcagctcgagtcacgaacttccctttttggag

SEQ ID NO: 7 - DNA, 2514 bp, Treponema pallidum
  CDS (1)..(2514)
atgctcaaaaaagccagtgccttcctaattgcaagttgttgtgtgatgtcgctggcgtgggcacaggcaaacgacaattggtacgagggaaagcctatctctgcgattagttttgaggggctcgaatatattgctcgcggccagttggacacgattttttctcaatacaagggacaaaagtggacctatgagctgtacctggagatactgcaaaaggtctatgaccttgagtacttttctgaagtttcgcctaaggcggtgcccaccgatccggagtatcagtatgtgatgctacagttcacggtaaaggagcgtccttcggtgaagggcatcaagatggtagggaacagccaaatccgcagtggggaccttttgtctaaaatcctcctgaaaaagggagacatttacaatgaagtaaagatgaaggtggaccaagagtcgctcaggcgtcattacctggaccagggctatgcggcggttaagatatcctgcgaggcaaaaactgaggcggggggcgtggtggtacagtttaccatccaggaaggtaagcagactgttgtctcgcggatacagtttaagggaaataaggcgtttaccgagtcggtgctcaagaaggtgctttccacgcaggaggcgcgttttttgaccagtggggtgttcaaggagaatgcgctggaagcggataaggcggcagtccactcatactatgcagagaggggatacattgacgcgcgggtagaaggcgtggcaaagacggttgataaaaaaactgacgccagtcgcaatctggttacgcttacgtacactgtggtggaaggtgagcagtaccgctacggcggggttaccattgtgggtaaccagatttttagcaccgaggagctgcaggcaaaaattaggctcaagcgcggggccatcatgaatatggtggcctttgagcagggctttcaggcgctggcggatgcgtattttgaaaacggatacacgtcaaattacctgaacaaagaagaacaccgggacacggcggagaaaacgctttcgtttaagatcacggtggtggagcgcgagcgcagccacgtcgagcacattatcattaagggaacgaagaatacaaaagacgaggttatcctgcgtgaaatgctgctgaaaccgggggatgtgttctctaagtcaaagtttacggatagcttgcgcaatctgttcaacctgcgctatttctcgtcgctggtgccggatgtgcggcccggctctgagcaggacctggtggacattatcctgaatgtggaggagcagtcgacggcaaacgtgcagtttggggtgacgttttctggggtgggggaggcaggcacgttcccgctttcgctcttttgtcagtgggaagaaaagaattttttgggaaaagggaatgaaatttcagtaaatgcaaccttggggtctgaggcgcagagcctgaagctcgggtatgtggagcgctggtttctgggctctccgctgacggtgggctttgactttgaacttacgcacaaaaatctctttgtgtaccgcgcgggttcatacggcaacgggctgccgcacccgtacacgagcagggagcagtgggctagttcccctgggctggcagaatcgtttcgcctcaagtattcgcgctttgagtccgccatcggcgcgcacaccgggtaccagtggtatccgcgctatgcggtcattagggtgaacgggggggtggactttcgggttgtaaagaatttttacgataaggataacaatcagcccttcgacctgaccgtaaaagagcagctgaactggaccagtatcaattcgttttggacgagcgtttcgtttgacgggcgtgactttgcgtacgacccgtccagcggctggtttttaggacagcgctgtacgttcaacgggctcgttccctttctcgaaaaagagcattcgtttcgctccgacaccaaggccgagttctacgttaccctgctcaattatccggtctctgccgtgtggaacttaaagtttgtcttggctttctacaccggtgtgtccgttcaaacgtattatggacggaggaaaagcgaaaacggaaagggcaacggggtgcggtccggcgcgctggtaatagacggcgtgctggtagggcgcgggtggagcgaagacgcaaagaaaaacaccggagacctgctgctccaccactggattgagttccgctggccgctggcgcacggcattgtgtcctttgactttttctttgatgcggcaatggtgtacaacatcgaaagtcagtccccaaacgggtcatcgtccgccagcagctccagcagcagcagtagtagtagcagtagaaccaccagctctgaaggactgtacaaaatgagctacggtccggggctgcgctttacattgccgcaatttccgttaaaattggcgttcgcaaacaccttcacgtcacccggcggcatcccaaaaacaaagaaaaattggaattttgtgttgtcgttcacggtaaataatttgtag

SEQ ID NO: 8 - protein, 837 aa, Treponema pallidum
MLKKASAFLIASCCVMSLAWAQANDNWYEGKPISAISFEGLEYIARGQLDTIFSQYKGQKWTYELYLEILQKVYDLEYFSEVSPKAVPTDPEYQYVMLQFTVKERPSVKGIKMVGNSQIRSGDLLSKILLKKGDIYNEVKMKVDQESLRRHYLDQGYAAVKISCEAKTEAGGVVVQFTIQEGKQTVVSRIQFKGNKAFTESVLKKVLSTQEARFLTSGVFKENALEADKAAVHSYYAERGYIDARVEGVAKTVDKKTDASRNLVTLTYTVVEGEQYRYGGVTIVGNQIFSTEELQAKIRLKRGAIMNMVAFEQGFQALADAYFENGYTSNYLNKEEHRDTAEKTLSFKITVVERERSHVEHIIIKGTKNTKDEVILREMLLKPGDVFSKSKFTDSLRNLFNLRYFSSLVPDVRPGSEQDLVDIILNVEEQSTANVQFGVTFSGVGEAGTFPLSLFCQWEEKNFLGKGNEISVNATLGSEAQSLKLGYVERWFLGSPLTVGFDFELTHKNLFVYRAGSYGNGLPHPYTSREQWASSPGLAESFRLKYSRFESAIGAHTGYQWYPRYAVIRVNGGVDFRVVKNFYDKDNNQPFDLTVKEQLNWTSINSFWTSVSFDGRDFAYDPSSGWFLGQRCTFNGLVPFLEKEHSFRSDTKAEFYVTLLNYPVSAVWNLKFVLAFYTGVSVQTYYGRRKSENGKGNGVRSGALVIDGVLVGRGWSEDAKKNTGDLLLHHWIEFRWPLAHGIVSFDFFFDAAMVYNIESQSPNGSSSASSSSSSSSSSSRTTSSEGLYKMSYGPGLRFTLPQFPLKLAFANTFTSPGGIPKTKKNWNFVLSFTVNNL

SEQ ID NO: 9 - protein, 413 aa, Artificial Sequence
  Amino acids 22-434 of the native Tp0326 peptide used in the Tp0453-Tp0326 chimeric construct
QANDNWYEGKPISAISFEGLEYIARGQLDTIFSQYKGQKWTYELYLEILQKVYDLEYFSEVSPKAVPTDPEYQYVMLQFTVKERPSVKGIKMVGNSQIRSGDLLSKILLKKGDIYNEVKMKVDQESLRRHYLDQGYAAVKISCEAKTEAGGVVVQFTIQEGKQTVVSRIQFKGNKAFTESVLKKVLSTQEARFLTSGVFKENALEADKAAVHSYYAERGYIDARVEGVAKTVDKKTDASRNLVTLTYTVVEGEQYRYGGVTIVGNQIFSTEELQAKIRLKRGAIMNMVAFEQGFQALADAYFENGYTSNYLNKEEHRDTAEKTLSFKITVVERERSHVEHIIIKGTKNTKDEVILREMLLKPGDVFSKSKFTDSLRNLFNLRYFSSLVPDVRPGSEQDLVDIILNVEEQSTAN

SEQ ID NO: 10 - DNA, 2124 bp, Artificial Sequence
  Tp0453-Tp0326 chimera
atgggcagcagccatcatcatcatcatcacagcagcggcctggtgccgcgcggcagccatatggcatcagttgatccgttgggtgttgtgggatctggagctgatgtgtatctctacttccctgtcgctgggaacgagaatttgatttcaaggatcatcgaaaatcacgaatcaaaagcggacatcaagaagatagtcgatcgaacaactgcggtctacggtgccttctttgcaagatccaaagaattcagactctttggatctggatcttacccctatgctttcacaaatcttattttctcgcgttcagatgggtgggcgagtacaaaaaccgaacacggtatcacatattacgagagcgagcatactgatgtctctattcctgctcctcatttctcttgtgtgatttttggatcgtctaaaagggaacgaatgagcaagatgttgagcagattagttaaccctgacagaccgcaattgcctcctagatttgaaaaggaatgcacgtcagaaggaacatcacaaactgtcgctttatacataaaaaatggaggtcatttcatcactaaactgttaaattttccacagttgaacctccctttaggtgctatggaactctatctcacagctcgtcgaaatgagtacctctacacactaagcctccaactaggaaatgccaagattaacttcccgatacagttcctgatttcacgagttttgaatgctcatattcatgtggagggagacagactgatcattgaggatggaactatctcggcggaacgtttggcctcggtaatctcctccctgtactctaaaaagggctcttctggtggaggtggcagtggtggaggaggaagcggcgggggtgggtcacaagcaaatgataactggtacgaaggtaaacctatatcagcaatatcattcgaggggctcgaatacatcgcacgtgggcagttagatacgatcttctcccagtataaagggcagaagtggacctacgaactttacctggagattcttcaaaaggtttacgatttggaatatttttctgaagtgtctcccaaagctgttcccactgatcctgaataccaatacgttatgttacaattcactgtgaaagaaaggccctcagttaaaggaattaagatggttgggaattctcagattaggagtggagatttgctcagtaaaattcttcttaaaaagggtgatatctataatgaggttaagatgaaagtggatcaagaaagtcttagaagacattacttggaccagggatacgctgcagtgaaaatttcttgtgaagctaagacagaggctggcggcgttgttgtccagttcaccatccaagaagggaagcaaacggttgtcagtagaatccagttcaaaggaaacaaggctttcaccgaaagtgtccttaagaaggtactaagcactcaagaagctagatttctaacctctggtgtgtttaaggagaatgctctagaggccgacaaagctgcagtgcattcttactatgctgaaagaggatatatcgatgctagggttgagggtgtggctaagacggttgataagaagacagatgcatctagaaatctcgtaaccttgacctacactgtggtcgagggagagcaataccgatacggtggagtcactatagtgggaaaccaaatattcagcacagaagaactacaagctaaaattagacttaaacgaggagctattatgaatatggttgcttttgagcagggctttcaagccctcgctgacgcatactttgaaaacggttacacaagtaactacttgaacaaggaagaacacagggacacagctgaaaaaacgttatcttttaaaattacagtagtggagagagaaagatctcatgttgaacatatcattattaaaggaactaagaatactaaagacgaggttatcctaagagagatgcttttgaaacccggggatgtgtttagtaaatctaaatttactgatagtttacgtaaccttttcaatttgagatacttcagtagcctggttccagatgtaaggccaggcagcgagcaagatttagtcgatattatactaaacgttgaagaacaatctacagcaaactgactcgag

SEQ ID NO: 11 - protein, 705 aa, Artificial Sequence
  Synthetic Construct
MGSSHHHHHHSSGLVPRGSHMASVDPLGVVGSGADVYLYFPVAGNENLISRIIENHESKADIKKIVDRTTAVYGAFFARSKEFRLFGSGSYPYAFTNLIFSRSDGWASTKTEHGITYYESEHTDVSIPAPHFSCVIFGSSKRERMSKMLSRLVNPDRPQLPPRFEKECTSEGTSQTVALYIKNGGHFITKLLNFPQLNLPLGAMELYLTARRNEYLYTLSLQLGNAKINFPIQFLISRVLNAHIHVEGDRLIIEDGTISAERLASVISSLYSKKGSSGGGGSGGGGSGGGGSQANDNWYEGKPISAISFEGLEYIARGQLDTIFSQYKGQKWTYELYLEILQKVYDLEYFSEVSPKAVPTDPEYQYVMLQFTVKERPSVKGIKMVGNSQIRSGDLLSKILLKKGDIYNEVKMKVDQESLRRHYLDQGYAAVKISCEAKTEAGGVVVQFTIQEGKQTVVSRIQFKGNKAFTESVLKKVLSTQEARFLTSGVFKENALEADKAAVHSYYAERGYIDARVEGVAKTVDKKTDASRNLVTLTYTVVEGEQYRYGGVTIVGNQIFSTEELQAKIRLKRGAIMNMVAFEQGFQALADAYFENGYTSNYLNKEEHRDTAEKTLSFKITVVERERSHVEHIIIKGTKNTKDEVILREMLLKPGDVFSKSKFTDSLRNLFNLRYFSSLVPDVRPGSEQDLVDIILNVEEQSTAN

SEQ ID NO: 12 - protein, 684 aa, Artificial Sequence
  Tp0453-Tp0326 chimera without pET28a sequence
ASVDPLGVVGSGADVYLYFPVAGNENLISRIIENHESKADIKKIVDRTTAVYGAFFARSKEFRLFGSGSYPYAFTNLIFSRSDGWASTKTEHGITYYESEHTDVSIPAPHFSCVIFGSSKRERMSKMLSRLVNPDRPQLPPRFEKECTSEGTSQTVALYIKNGGHFITKLLNFPQLNLPLGAMELYLTARRNEYLYTLSLQLGNAKINFPIQFLISRVLNAHIHVEGDRLIIEDGTISAERLASVISSLYSKKGSSGGGGSGGGGSGGGGSQANDNWYEGKPISAISFEGLEYIARGQLDTIFSQYKGQKWTYELYLEILQKVYDLEYFSEVSPKAVPTDPEYQYVMLQFTVKERPSVKGIKMVGNSQIRSGDLLSKILLKKGDIYNEVKMKVDQESLRRHYLDQGYAAVKISCEAKTEAGGVVVQFTIQEGKQTVVSRIQFKGNKAFTESVLKKVLSTQEARFLTSGVFKENALEADKAAVHSYYAERGYIDARVEGVAKTVDKKTDASRNLVTLTYTVVEGEQYRYGGVTIVGNQIFSTEELQAKIRLKRGAIMNMVAFEQGFQALADAYFENGYTSNYLNKEEHRDTAEKTLSFKITVVERERSHVEHIIIKGTKNTKDEVILREMLLKPGDVFSKSKFTDSLRNLFNLRYFSSLVPDVRPGSEQDLVDIILNVEEQSTAN

SEQ ID NO: 13 - protein, 9 aa, Artificial Sequence
  Myc tag
EQKLISEED

SEQ ID NO: 14 - protein, 8 aa, Artificial Sequence
  FLAG tag
DYKDDDDK

SEQ ID NO: 15 - protein, 15 aa, Artificial Sequence
  S tag
KETAAAKFERQHMDS

SEQ ID NO: 16 - protein, 20 aa, Artificial Sequence
  exemplary linker
GGGGSGGGGSGGGGSGGGGS

SEQ ID NO: 17 - protein, 15 aa, Artificial Sequence
  exemplary linker sequence
RGRGRGRGRSRGGGS